(19) United States
(12) Patent Application Publication
(10) Pub. No.: US 2021/0238240 A1
(43) Pub. Date: Aug. 5, 2021

(54) METHODS AND COMPOSITIONS FOR RNA EXPRESSION OF MYC INHIBITORS

(71) Applicant: Helix Nanotechnologies, Inc., Walnut, CA (US)

(72) Inventors: Nikolai Eroshenko, Boston, MA (US); Nikhil Dhar, Boston, MA (US); Taylor Gill, Cambridge, MA (US); Marianna Keaveney, Walpole, MA (US); Hannu Rajaniemi, San Francisco, CA (US)

(21) Appl. No.: 17/269,787

(22) PCT Filed: Aug. 20, 2019
(86) PCT No.: PCT/US2019/047243

§ 371 Date: Feb. 19, 2021

Related U.S. Application Data
(60) Provisional application No. 62/720,105, filed on Aug. 20, 2018.

(51) Int. Cl. C07K 14/47; C07K 14/005; A61P 35/00

(52) U.S. Cl. CPC C07K 14/4702; A61K 38/00; A61P 35/00; C07K 14/005

(57) ABSTRACT

Nucleic acid expression systems for delivery of RNA oligonucleotides to target cells and methods of using the same are provided herein. For example, in some embodiments, a nucleic acid expression system comprises: (i) an RNA oligonucleotide comprising a payload sequence (e.g., a Myc inhibitor), and (ii) an RNA oligonucleotide comprising a sequence that encodes a US11 polypeptide. Provided herein are also pharmaceutical compositions comprising an RNA oligonucleotide comprising a payload sequence that encodes a dominant negative variant of a Myc polypeptide or portions thereof, and a pharmaceutically acceptable carrier.

### CROSS-REFERENCE TO RELATED APPLICATIONS

This application claims the benefit of U.S. Provisional Application No. 62/720,105 filed Aug. 20, 2018, the contents of which are hereby incorporated herein in their entirety.

### BACKGROUND

In recent years, research progress has been made to gene therapy or RNA therapy for treating or improving a particular condition or disease. Existing gene-encoded therapeutics are generally based on one of three approaches: engineered viruses, non-viral DNA vectors, and modified RNAs. However, each of these approaches currently have significant technological limitations.

### SUMMARY

The present disclosure provides technologies for delivering RNA oligonucleotides to a subject or a target cell. The present disclosure recognizes that at least one challenge associated with RNA oligonucleotide delivery includes developing RNA chemistries that are not recognized by myriad innate immune sensors while still efficiently recognized by a translational machinery in a cell. The present disclosure addresses at least this challenge and provides nucleic acid expression systems and compositions for delivery of an RNA (e.g., mRNA) oligonucleotide comprising a payload sequence that encodes a Myc inhibitor with an RNA (e.g., mRNA) oligonucleotide comprising a sequence that encodes a US11 polypeptide. Methods for using such nucleic acid expression systems and compositions are also provided herein. The present disclosure also provides nucleic acid expression systems and compositions for delivery of an RNA (e.g., mRNA) oligonucleotide comprising a payload sequence that encodes a Myc inhibitor. Some of the advantages provided by nucleic acid expression systems, compositions, and methods described herein include, but are not limited to, increasing expression of an RNA (e.g., mRNA) oligonucleotide comprising a sequence that encodes, e.g., a Myc inhibitor; reducing non-specific toxicity induced by RNA (e.g., mRNA) oligonucleotides (e.g., comprising a sequence that encodes a Myc inhibitor); and/or reducing innate immunity-trigger suppression of protein translation and/or RNA degradation in target cells.

In one aspect, provided herein is a nucleic acid expression system comprising: (i) an RNA oligonucleotide comprising a payload sequence that encodes a Myc inhibitor, and (ii) an RNA oligonucleotide comprising a sequence that encodes a US11 polypeptide.

In some embodiments, an RNA oligonucleotide comprising a payload sequence that encodes a Myc inhibitor is a synthetic RNA oligonucleotide. In some embodiments, an RNA oligonucleotide comprising a sequence that encodes a US11 polypeptide is a synthetic RNA oligonucleotide.

In some embodiments, an RNA oligonucleotide comprising a payload sequence that encodes a Myc inhibitor is a messenger RNA (mRNA) oligonucleotide. In some embodiments, an RNA oligonucleotide comprising a sequence that encodes a US11 polypeptide is a mRNA oligonucleotide.

In some embodiments, a US11 polypeptide encoded by a sequence of an RNA oligonucleotide is or includes an RNA binding domain of a US11 polypeptide. In some embodiments, a US11 polypeptide comprises the sequence of SEQ ID NO.: 1 or SEQ ID NO: 2.

In some embodiments, a Myc inhibitor encoded by a payload sequence of an RNA oligonucleotide reduces expression and/or activity of Myc. An exemplary Myc inhibitor includes, but is not limited to a dominant negative variant of a Myc polypeptide.

In some embodiments, a Myc inhibitor is or comprises a variant of at least one domain of a Myc polypeptide, e.g., at least one or more of a basic helix-loop-helix DNA-binding domain, a leucine zipper domain, and a transactivation domain of a Myc polypeptide.

In some embodiments, a Myc inhibitor is or comprises a variant of a leucine zipper domain of a Myc polypeptide, and may optionally include a basic helix-loop-helix DNA-binding domain of a Myc polypeptide or a variant thereof. In some embodiments, a Myc inhibitor can lack a transactivation domain of a Myc polypeptide.

In some embodiments, a Myc inhibitor can dimerize with a Myc polypeptide (e.g., a wild-type Myc polypeptide). Additionally or alternatively, a Myc inhibitor can dimerize with a Max polypeptide (e.g., a wild-type Max polypeptide) to form a dimer, e.g., which can then bind to an E-box sequence to form a complex that does not promote transcription.

In some embodiments, a Myc inhibitor does not interfere with Myc/Miz-1 dimerization and/or transcriptional repression.

In some embodiments, a Myc inhibitor is or comprises an OmoMYC polypeptide.

In some embodiments, a Myc inhibitor is or comprises an amino acid sequence that is at least 90%, 91%, 92%, 93%, 94%, 95%, 96%, 97%, 98%, or 100% identical to the sequence of SEQ ID NO.: 3. In some embodiments, a Myc inhibitor is or comprises an amino acid sequence that is based on the sequence of SEQ ID NO.: 3 and includes 0-10 amino acid modifications to the sequence of SEQ ID NO.: 3. In some embodiments, a Myc inhibitor is or comprises the amino acid sequence of SEQ ID NO.: 3.

Compositions comprising a nucleic acid expression system according to any one of the embodiments described herein are also provided herein. In some embodiments, a composition can be or comprises a pharmaceutical composition, which may optionally further comprise a pharmaceutically acceptable carrier.

Another aspect provided herein relates to cells comprising a nucleic acid expression system according to any one of the embodiments described herein. An exemplary cell may include, but is not limited to, a cancer cell.

Methods for using any embodiments of nucleic acid expression systems, compositions (e.g., pharmaceutical compositions), and/or cells are also provided herein. In some embodiments, a method comprises: (a) contacting a target cell with an RNA oligonucleotide comprising a payload sequence that encodes a Myc inhibitor; and (b) contacting the target cell with an RNA oligonucleotide comprising a sequence that encodes a US11 polypeptide. In some embodiments, an RNA (e.g., mRNA) oligonucleotide can comprise any sequence that encodes a Myc inhibitor described herein. In some embodiments, an RNA (e.g., mRNA) oligonucleotide can comprise any sequence that encodes a US11 polypeptide described herein.

In some embodiments, a method described herein can be used for enhancing expression and/or activity of a payload sequence that encodes a Myc inhibitor in a target cell. For example, in some embodiments, expression and/or activity of a payload sequence that encodes a Myc inhibitor in a target cell can be enhanced by at least 30% or more, as compared to the expression and/or activity of the payload sequence in a target cell in the absence of an RNA oligonucleotide comprising a sequence that encodes the US11 polypeptide.

In some embodiments, a method described herein can be used for enhancing viability of a target cell upon contacting with an RNA oligonucleotide comprising a payload sequence that encodes a Myc inhibitor and an RNA oligonucleotide comprising a sequence that encodes a US11 polypeptide. For example, in some embodiments, viability of a target cell upon contacting with an RNA oligonucleotide comprising a payload sequence that encodes a Myc inhibitor and an RNA oligonucleotide comprising a sequence that encodes a US11 polypeptide can be enhanced by at least 30% or more, as compared to the viability of a target cell upon contacting with an RNA oligonucleotide comprising the payload sequence in the absence of the RNA oligonucleotide that encodes a US11 polypeptide.

In some embodiments, a method described herein can be used for reducing non-specific toxicity induced in a target cell by an RNA oligonucleotide comprising a payload sequence that encodes a Myc inhibitor. For example, in some embodiments, non-specific toxicity induced in a target cell by an RNA oligonucleotide comprising a payload sequence that encodes a Myc inhibitor can be reduced by at least 30% or more, as compared to the non-specific toxicity induced in a target cell by an RNA oligonucleotide comprising the payload sequence in the absence of an RNA oligonucleotide comprising a sequence that encodes a US11 polypeptide.

In some embodiments, a target cell may be previously contacted at least once by one or more oligonucleotides.

A target cell in methods described herein may be contacted with an RNA oligonucleotide comprising a payload sequence that encodes a Myc inhibitor and an RNA oligonucleotide comprising a sequence that encodes a US11 polypeptide concurrently or separately. In some embodiments, a target cell is contacted with an RNA oligonucleotide comprising a payload sequence that encodes a Myc inhibitor and an RNA oligonucleotide comprising a sequence that encodes a US11 polypeptide separately, e.g., within 24 hours or less.

In some embodiments, a target cell (e.g., a cancer cell) in a method described herein is present in a subject. In some such embodiments, an RNA oligonucleotide comprising a payload sequence that encodes a Myc inhibitor can be administered to a subject in need thereof, e.g., a subject having cancer. Additionally or alternatively, an RNA oligonucleotide comprising a sequence that encodes a US11 polypeptide can be administered to a subject in need thereof, e.g., a subject having cancer.

In some embodiments where a target cell in a method described herein is a cancer cell, the method can be used for attenuating a cancer cell. Accordingly, a method of attenuating a cancer cell comprising (a) contacting a cancer cell with an RNA oligonucleotide comprising a payload sequence that encodes a Myc inhibitor; and (b) contacting the cancer cell with an RNA oligonucleotide comprising a sequence that encodes a US11 polypeptide is also provided herein.

In some embodiments, any Myc inhibitor described herein can be encoded in a payload sequence of an RNA oligonucleotide that is delivered to a cancer cell. In some embodiments, any US11 polypeptide described herein can be encoded in a sequence of an RNA oligonucleotide that is delivered to a cancer cell.

A cancer cell in some embodiments of a method described herein may be from, for example, leukemia, neuroblastoma, lymphoma, breast cancer, colon cancer, lung cancer, ovarian cancer, thymoma, germ cell tumor, myeloma, melanoma, rectal cancer, stomach cancer, pancreatic cancer, testicular cancer, skin cancer, sarcoma, or brain cancer.

Also within the scope of the present disclosure relates to a pharmaceutical composition comprising: (i) an RNA oligonucleotide comprising a payload sequence that encodes a dominant negative variant of a Myc polypeptide, and (ii) a pharmaceutically acceptable carrier. In some embodiments, a dominant negative variant of a Myc polypeptide described herein can be encoded by a payload sequence of an RNA oligonucleotide.

These, and other aspects encompassed by the present disclosure, are described in more detail below and in the claims.

### BRIEF DESCRIPTION OF THE DRAWINGS

FIG. 1 depicts expression of a model payload sequence when an RNA oligonucleotide comprising a payload sequence is delivered to target cells in the presence of various amounts of an RNA oligonucleotide comprising a sequence that encodes a US11 polypeptide. Luciferase luminescence (y-axis) indicates expression of a model payload sequence (e.g., luc2).

FIGS. 2A-2C depict viability of cells upon repeated transfections with an RNA oligonucleotide comprising a payload sequence that encodes a Myc inhibitor with or without an RNA oligonucleotide comprising a sequence that encodes a US11 polypeptide. FIG. 2A shows cell viability after a first transfection with RNA oligonucleotides as indicated according to one embodiment described herein. FIG. 2B shows cell viability after a second transfection with RNA oligonucleotide as indicated according to one embodiment described herein. FIG. 2C shows cell viability after a third transfection with RNA oligonucleotides as indicated according to one embodiment described herein.

FIGS. 3A-3C depict viability of cells upon repeated transfections with an exemplary RNA oligonucleotide comprising a payload sequence that encodes a Myc inhibitor with or without an exemplary RNA oligonucleotide comprising a sequence that encodes a US11 polypeptide. FIG. 3A shows cell viability after a first transfection with RNA oligonucleotides as indicated according to another embodiment described herein. FIG. 3B shows cell viability after a second transfection with RNA oligonucleotide as indicated according to another embodiment described herein. FIG. 3C shows cell viability after a third transfection with RNA oligonucleotides as indicated according to another embodiment described herein.

### CERTAIN DEFINITIONS

About or approximately: As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to a subject to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Co-delivery: As used herein, the term “co-delivery” refers to use of both an RNA oligonucleotide comprising a payload sequence that encodes a Myc inhibitor and an RNA oligonucleotide comprising a sequence that encodes a US11 polypeptide to deliver a payload sequence into a target cell. The combined use of an RNA oligonucleotide comprising a payload sequence that encodes a Myc inhibitor and an RNA oligonucleotide comprising a sequence that encodes a US11 polypeptide may be performed concurrently or separately (e.g., sequentially in any order). In some embodiments of a pharmaceutical composition described herein, both an RNA oligonucleotide comprising a payload sequence that encodes a Myc inhibitor and an RNA oligonucleotide comprising a sequence that encodes a US11 polypeptide may be combined in one pharmaceutically-acceptable carrier, or they may be placed in separate carriers and delivered to a target cell (e.g., a cancer cell) or administered to a subject at different times. Each of these situations is contemplated as falling within the meaning of “co-delivery” or “co-administration” or “combination,” provided that both an RNA oligonucleotide comprising a payload sequence that encodes a Myc inhibitor and an RNA oligonucleotide comprising a sequence that encodes a US11 polypeptide are delivered or administered sufficiently close in time that there is at least some temporal overlap in biological effect(s) generated by both RNA oligonucleotides on a target cell or a subject being treated.

Delivery/contacting: As used interchangeably herein, the term “delivery,” “delivering,” or “contacting” refers to introduction of an RNA oligonucleotide (e.g., comprising a payload sequence that encodes a Myc inhibitor or comprising a sequence encoding a US11 polypeptide) into a target cell (e.g., cytosol of a target cell). A target cell can be cultured in vitro or ex vivo or be present in a subject (in vivo). Methods of introducing an RNA oligonucleotide into a target cell can vary with in vitro, ex vivo, or in vivo applications. In some embodiments, an RNA oligonucleotide can be introduced into a target cell in a cell culture by in vitro transfection. In some embodiments, an RNA oligonucleotide can be introduced into a target cell via delivery vehicles (e.g., nanoparticles, liposomes, and/or complexation with a cell-penetrating agent). In some embodiments, an RNA oligonucleotide can be introduced into a target cell in a subject by administering an RNA oligonucleotide to a subject.

Dominant negative: As used herein, a “dominant negative” polypeptide is an inactive variant of a protein or polypeptide (e.g., a Myc inhibitor), which, by interacting with the cellular machinery, displaces an active protein from its interaction with the cellular machinery and/or competes with the active protein, thereby reducing the effect of the active protein. For example, a dominant negative receptor which binds a ligand, but does not transmit a signal in response to binding of the ligand, can reduce the biological effect of expression of the ligand. Likewise, a dominant negative catalytically-inactive kinase which interacts normally with one or more target proteins, but does not phosphorylate the target proteins, can reduce phosphorylation of the target proteins in response to a cellular signal. Similarly, a dominant negative transcription factor which binds to a promoter site in the control region of a gene, but does not increase gene transcription, can reduce the effect of a normal transcription factor, by occupying promoter binding sites without increasing transcription.

Inhibitor: As used herein, the term “inhibitor” refers to a molecule whose presence, level, or degree correlates with decreased level or activity of a target. In some embodiments, an inhibitor may be act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

Non-specific toxicity: In context of introduction of an RNA oligonucleotide, e.g., an RNA oligonucleotide comprising a Myc inhibitor-encoding payload sequence, into a target cell, the term “non-specific toxicity” refers to cell toxicity induced by an RNA oligonucleotide independent of a function and/or activity of a Myc inhibitor-encoding payload sequence. For example, when an RNA oligonucleotide comprising a non-cytotoxic payload sequence causes comparable cell death (an exemplary indicator of cell toxicity) to that caused by an RNA oligonucleotide comprising a cytotoxic payload sequence, the cell death (or cell toxicity) is nonspecific because it is independent of the cytotoxic nature of a payload sequence. In some embodiments, “non-specific toxicity” also refers to cell toxicity induced in any cells including, e.g., both target and non-target cells (e.g., normal healthy cells), rather than induced in target cells (e.g., cancer cells) only.

Nucleic acid/Oligonucleotide: As used herein, the terms “nucleic acid” and “oligonucleotide” are used interchangeably, and refer to a polymer of at least 3 nucleotides or more. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of an RNA oligonucleotide, a certain number of nucleotides refers to the number of nucleotides on a single strand, e.g., of an RNA oligonucleotide.

Polypeptide: The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

RNA oligonucleotide: As used herein, the term “RNA oligonucleotide” refers to an oligonucleotide of ribonucleotides. In some embodiments, an RNA oligonucleotide is single stranded. In some embodiments, an RNA oligonucleotide is double stranded. In some embodiments, an RNA oligonucleotide comprises both single and double stranded portions. In some embodiments, an RNA oligonucleotide can comprise a backbone structure as described in the definition of “Nucleic acid/Oligonucleotide” above. An RNA oligonucleotide can be a regulatory RNA (e.g., siRNA, microRNA, etc.), or a messenger RNA (mRNA) oligonucleotide. In some embodiments where an RNA oligonucleotide is a mRNA oligonucleotide, an RNA oligonucleotide typically comprises at its 3′ end a poly(A) region. In some embodiments where an RNA oligonucleotide is a mRNA oligonucleotide, an RNA oligonucleotide typically comprises at its 5′ end an art-recognized cap structure, e.g., for recognizing and attachment of a mRNA to a ribosome to initiate translation.

Subject: As used herein, the term “subject” refers an organism, typically a mammal (e.g., a human). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is an individual (e.g., a human) who has undergone an RNA oligonucleotide therapy or a gene therapy at least once or more. In some embodiments, a subject is an individual (e.g., a human) who is undergoing an RNA oligonucleotide therapy or a gene therapy.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide.

Standard techniques may be used for recombinant DNA, oligonucleotide synthesis, e.g., mRNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2nd ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

Gene-encoded therapeutics can be delivered using either DNA-based vectors, such as plasmids or minicircles, or with recombinant viral vectors, such as lentiviruses or adeno-associated virus (Yin et al., (2014) Nature Reviews Genetics 15: 541-55; Kotterman et al., (2015) Annual Review of Biomedical Engineering 17: 63-89, each of which is incorporated by reference in its entirety). While messenger RNAs (mRNAs) may be a competing delivery modality to address some technical deficiencies with both viral and non-viral DNA vectors (Sahin et al. (2014) Nature Reviews Drug Discovery 13: 759-80 which is incorporated by reference in its entirety), there are challenges of delivering mRNAs into subjects, e.g., high immunogenicity associated with foreign RNAs. For example, since many viruses generate either fully double stranded RNA (dsRNA) or single-stranded RNA (ssRNA) with secondary structures during their life cycles, human beings have evolved sophisticated innate immune sensors to enable both professional antigen-presenting cells (APCs) and non-immune cells to detect double-stranded and improperly capped RNAs.

While chemically modified mRNAs were made using non-standard base chemistries to reduce their immunogenicity (Karikó et al. (2005) Immunity 23: 165-175; Svitkin et al. (2017) Nucleic Acids Research 45:6023-6036, each of which is incorporated by reference in its entirety), concerns with residual immune response that precludes repeated dosing and/or high-level dosing remain. At least one challenge includes developing RNA chemistries that are not recognized by myriad innate immune sensors while still efficiently recognized by a translational machinery in a cell. Accordingly, there remains a need to develop methods and compositions for improving RNA therapeutics.

The present disclosure is based, at least in part, on an unexpected discovery that co-delivery (e.g., to a subject or target cell) of an RNA (e.g., an messenger RNA (mRNA)) oligonucleotide comprising a sequence that encodes a US11 polypeptide (among a large set of candidate immunomodulatory polypeptides, e.g., viral immune suppressors) with an RNA (e.g., mRNA) encoding a Myc inhibitor results in increased expression of the Myc inhibitor. The present disclosure recognizes that a US11 polypeptide may reduce innate immunity-triggered suppression of protein translation and/or RNA degradation. A reduction in innate immunity-triggered suppression of protein translation and/or RNA degradation can, in turn, improve expression of a Myc inhibitor from a co-delivered RNA (e.g., mRNA) oligonucleotide in target cells. The present disclosure also encompasses the surprising discovery that delivery of an RNA (e.g., mRNA) oligonucleotide comprising a sequence that encodes a US11 polypeptide to, e.g., a subject or target cell, can reduce non-specific toxicity induced by RNA (e.g., mRNA) oligonucleotides. The present disclosure also encompasses the surprising discovery that co-delivery of an RNA (e.g., mRNA) oligonucleotide comprising a sequence that encodes a US11 polypeptide with an RNA (e.g., mRNA) oligonucleotide comprising a payload sequence that encodes a Myc inhibitor can reduce non-specific toxicity induced by RNA (e.g., mRNA) oligonucleotides, e.g., the RNA (e.g., mRNA) encoding a Myc inhibitor. Further, the present disclosure encompasses the surprising discovery that co-delivery of an RNA (e.g., mRNA) oligonucleotide comprising a sequence that encodes a US11 polypeptide with an RNA (e.g., mRNA) oligonucleotide comprising a payload sequence that encodes a Myc inhibitor, at doses that were shown to be non-toxic with a negative control payload (i.e., a payload that does not modulate expression of any gene), induces apoptosis/death and/or growth arrest of cancer cells, thereby enabling selective attenuation cancer cells (e.g., Kras or Myc-expressing cancer cells). The present disclosure provides improved compositions including and uses of an RNA (e.g., mRNA) oligonucleotide comprising a payload sequence that encodes a Myc inhibitor, e.g., for treatment of cancer. The present disclosure also provides compositions including an RNA (e.g., mRNA) oligonucleotide comprising a sequence that encodes a US11 polypeptide that can be delivered more than once to a subject or target cells, e.g., to improve expression and/or activity of, e.g., an RNA (e.g., mRNA) oligonucleotide comprising a payload sequence that encodes a Myc inhibitor without substantially increasing non-specific toxicity induced by RNA oligonucleotides.

Accordingly, the present disclosure provides nucleic acid expression systems and compositions for delivery of an RNA (e.g., mRNA) oligonucleotide comprising a payload sequence that encodes a Myc inhibitor. The present disclosure provides nucleic acid expression systems and compositions for delivery of an RNA (e.g., mRNA) oligonucleotide comprising a payload sequence that encodes a Myc inhibitor with an RNA (e.g., mRNA) oligonucleotide comprising a sequence that encodes a US11 polypeptide. Methods for using such nucleic acid expression systems and compositions are also provided herein.

### I. Nucleic Acid Expression Systems

In one aspect, the present disclosure provides nucleic acid expression systems for expressing RNA oligonucleotides in cells. In some embodiments, a nucleic acid expression system includes an RNA oligonucleotide comprising a payload sequence that encodes a Myc inhibitor and an RNA oligonucleotide comprising a sequence that encodes a US11 polypeptide.

In some embodiments, RNA oligonucleotides (e.g., comprising a payload sequence that encodes a Myc inhibitor and/or encoding a US11 polypeptide) of any aspects described herein are synthetic RNA oligonucleotides. For example, in some embodiments, an RNA oligonucleotide comprising a payload sequence is a synthetic RNA oligonucleotide. In some embodiments, an RNA oligonucleotide comprising a sequence that encodes a US11 polypeptide is a synthetic RNA oligonucleotide. Synthetic RNA oligonucleotides can be produced by any methods known in the art. For example, in some embodiments where synthetic RNA oligonucleotides are synthetic mRNA oligonucleotides, they can be produced, e.g., by in vitro transcription of a cDNA template, typically plasmid DNA (pDNA), using an RNA polymerase, e.g., a bacteriophage RNA polymerase.

In some embodiments, RNA oligonucleotides (e.g., comprising a payload sequence that encodes a Myc inhibitor and/or encoding a sequence that encodes a US11 polypeptide) of any aspects described herein are messenger RNA (mRNA) oligonucleotides. For example, in some embodiments, an RNA oligonucleotide comprising a payload sequence is a mRNA oligonucleotide. In some embodiments, an RNA oligonucleotide comprising a sequence that encodes a US11 polypeptide is a mRNA oligonucleotide.

In some embodiments, a nucleic acid expression system includes at least one RNA oligonucleotide comprising a payload sequence as described herein and least one RNA oligonucleotide comprising a sequence that encodes a US11 polypeptide as described herein. In some embodiments, a nucleic acid expression system includes 1, 2, 3, 4, 5, 6, 7, 8, 9, or 10 RNA oligonucleotides comprising a payload sequence. In some embodiments, a nucleic acid expression system includes 1, 2, 3, 4, 5, 6, 7, 8, 9, or 10 oligonucleotides that encoding a US11 polypeptide.

US11 Polypeptides

The present disclosure demonstrates, among other things, that use of a US11 polypeptide that suppresses or inhibits innate immunity pathways of host cells can improve expression of an RNA oligonucleotide comprising a payload sequence introduced into the host cells, e.g., by inhibiting host immunity-triggered suppression of protein translation and mRNA degradation, enhancing the expression and/or activity of a payload oligonucleotide in host cells, reducing non-specific toxicity in host cells induced by a payload oligonucleotide, and/or increasing viability of cells upon introduction of a payload oligonucleotide.

Non-immune somatic cells detect the presence of foreign RNA (e.g., mRNA) using sensor proteins, including, e.g., but not limited to retinoic acid inducible gene I (RIG-1), melanoma differentiation-associated antigen 5 (MDA5), protein kinase R (PKR) and 2′-5′-oligoadenylate synthetase (OAS) (Sahin et al. (2014) Nature Reviews Drug Discovery 13: 759-780, which is incorporated by reference in its entirety). Innate immune activation by RIG-I, which senses 5′ triphosphates characteristic of uncapped viral transcripts, and MDA5, which detects long dsRNA, can be ameliorated, e.g., via using non-standard base chemistries to make mRNA therapeutics (Karikó et al. (2011) Immunity 23: 165-175; Mu et al. (2018) Nucleic Acids Research 46(10):5239-5249, each of which is incorporated by reference in its entirety). Short stretches of dsRNA, sensed by PKR and the OAS proteins, are more difficult to evade host innate immunity than long dsRNA due to the presence of structured mRNA in many naturally occurring human transcripts (Mortimer et al. (2014) Nat Rev Genet. 15:469-79, which is incorporated by reference in its entirety).

In some embodiments, a US11 polypeptide reduces expression and/or activity at least one or more of RIG-I, MDA5, PKR, and OAS. In some embodiments, a US11 polypeptide is an inhibitor of RIG-I. In some embodiments, a US11 polypeptide is an inhibitor of MDA5. In some embodiments, a US11 polypeptide is an inhibitor of PKR. In some embodiments, a US11 polypeptide is an inhibitor of OAS. In some embodiments, a US11 polypeptide is an inhibitor of RIG-I and MDA5. In some embodiments, a US11 polypeptide is an inhibitor of PKR and OAS. In some embodiments, a US11 polypeptide is an inhibitor of RIG-I, MDA5, PKR, and OAS.

In some embodiments, a US11 polypeptide is obtained or derived from dsRNA viruses (e.g., Adenoviruses, Herpesviruses, Poxviruses), ssDNA viruses (e.g., Parvoviruses), dsRNA viruses (e.g., Reoviruses), (+)ssRNA viruses (single-stranded positive-sense RNA viruses, e.g., Picornaviruses, Togaviruses), (−)ssRNA viruses (single-stranded negative-antisense RNA viruses, e.g., Orthomyxoviruses, Rhabdoviruses), ssRNA-RT viruses (single-stranded positive-sense RNA viruses with reverse transcriptase (RT) and/or DNA intermediates in life-cycle (e.g., Retroviruses), dsDNA-RT viruses (double-stranded reverse transcribing viruses with RNA intermediates in life-cycle, e.g., Hepadnaviruses.

In some embodiments, a US11 polypeptide is derived or obtained from dsRNA viruses. For example, in some embodiments, a US11 polypeptide is or includes a herpesvirus polypeptide, e.g., a herpes simplex virus (HSV) polypeptide. In some embodiments, a viral US11 polypeptide is or includes a herpes simplex virus type 1 (HSV-1) polypeptide, e.g., a HSV-1 tegument polypeptide.

In some embodiments, a US11 polypeptide is or includes an RNA-binding domain of a US11 polypeptide. In some embodiments, a US11 polypeptide is or includes a US11 polypeptide. In some embodiments, a US11 polypeptide (e.g., including an RNA-binding domain of US11 polypeptide) can inhibit at least one (including, e.g., one, two, three, or four) of RIG-I, MDA5, PKR, and OAS RNA sensors present in non-immune cells. In some embodiments, a US11 polypeptide (e.g., including an RNA-binding domain of a US11 polypeptide) can bind to and block the phosphorylation of PKR (Cassady & Gross (2002) Journal of Virology 76:2029-35, which is incorporated by reference in its entirety), directly interact with and inhibits MDA5 and RIG-I (Xing et al. (2012) Journal of Virology 86: 3528-3540, which is incorporated by reference in its entirety), and/or block OAS-dsRNA binding (Sanchez & Mohr (2007) Journal of Virology 81: 3455-3464, which is incorporated by reference in its entirety). In some embodiments, a US11 polypeptide (e.g., including an RNA-binding domain of a US11 polypeptide) can inhibit PKR and/or OAS in mitochondrial antiviral signaling (MAVS) knock-out (KO) cells. In some embodiments, a US11 polypeptide (e.g., including an RNA-binding domain of a US11 polypeptide) can inhibit PKR-driven protein degradation and/or OAS-drive RNAse activity.

In some embodiments, a US11 polypeptide (e.g., including an RNA-binding domain of a US11 polypeptide) includes an amino acid sequence that is based on the corresponding domain(s) of tegument US11 polypeptide from HSV-1. For example, a US 11 polypeptide (alternatively called y134.5) is encoded in two copies by the herpes simplex virus type 1 (HSV-1) genome, and has a uniquely broad role in the suppression of innate immunity (Chou et al. (1990) Science 250: 1262-6, which is incorporated by reference in its entirety). This immune suppression function is desirable in HSV-1 because despite being a dsDNA virus, more than half of the HSV-1 genome forms dsRNA side-products (Jacquemont & Roizman (1975) Journal of Virology 15: 707-13, which is incorporated by reference in its entirety).

In some embodiments, a US11 polypeptide (e.g., including an RNA-binding domain of a US11 polypeptide) can be a US11 homologue from other herpes viruses or viral families, which may have acquired US11-type proteins via horizontal gene transfer.

In some embodiments, a US11 polypeptide comprises the sequence of SEQ ID NO: 1, which is set forth below:

(SEQ ID NO: 1)
MSQTQPPAPVGPGDPDVYLKGVPSAGMHPRGVHAPRGHPRMISGPPQRGD
NDQAAGQCGDSGLLRVGADTTISKPSEAVRPPTIPRTPRVPREPRVPRPP
REPREPRVPRAPRDPRVPRDPRDPRQPRSPREPRSPREPRSPREPRTPRT
PREPRTARGSV

In some embodiments, a US11 polypeptide comprises the sequence of SEQ ID NO: 2, which is set forth below:

(SEQ ID NO: 2)
MPRVPRPPREPREPRVPRAPRDPRVPRDPRDPRQPRSPREPRSPREPRSP
REPRTPRTPREPRTARGSV.

US11 polypeptides described herein are delivered via RNA oligonucleotides. In some embodiments, an RNA oligonucleotide that encodes a US11 polypeptide described herein is a mRNA oligonucleotide. Delivering US11 polypeptides to target cells via mRNA oligonucleotides may be more advantageous in certain aspects than protein-based delivery. For example, some proteins cannot traverse the cellular membrane due to their large size. Additionally, in the context of delivery of RNA oligonucleotides, US11 polypeptides delivered via mRNA oligonucleotides can have an advantage of matching expression kinetics and cellular localization of payload mRNA oligonucleotides.

Payload Sequence Encoding a Myc Inhibitor

A payload sequence of any aspects described herein encodes a Myc inhibitor. Examples of Myc inhibitors include, but are not limited to Myc-binding peptides, anti-Myc antibodies or antigen-binding fragments thereof, and dominant negatives of Myc in an amount effective to reduce expression and/or activity of Myc.

In some embodiments, a Myc inhibitor encoded by a payload oligonucleotide (e.g., a payload RNA oligonucleotide) reduces expression and/or activity of Myc (e.g., m-Myc, N-Myc, and/or L-Myc) by at least 30% or more, including, e.g., at least 40%, at least 50%, at least 60%, at least 70%, at least 80%, at least 90%, or more, as compared to the expression and/or activity of Myc in the absence of the Myc inhibitor-encoding payload oligonucleotide. In some embodiments, a Myc inhibitor encoded by a payload oligonucleotide (e.g., a payload RNA oligonucleotide) comprises a dominant negative variant of a Myc polypeptide.

In some embodiments, a Myc inhibitor (e.g., a dominant negative variant) of Myc is or comprises a variant of at least one domain of a Myc polypeptide, e.g., a basic helix-loop-helix DNA-binding domain, a leucine zipper domain, and a transactivation domain of a Myc polypeptide. A Myc polypeptide or transcription factor contains a basic helix-loop-helix DNA-binding domain, which enables it to bind to E-boxes throughout the genome to drive transcription of target genes, and a leucine zipper domain, enabling it to dimerize with other leucine zipper-containing transcription factor proteins (Cowling & Cole (2006) Seminars in Cancer Biology 16: 242-252). In order to bind E-boxes and drive transcription of target genes, Myc dimerizes with its obligate heterodimerization partner Max. Myc may also bind to Miz-1, and the Myc/Miz-1 heterodimer binds to non-E-box sequences to purportedly repress transcription (Wanzel et al. (2003) Trends in Cell Biology 13: 146-150, which is incorporated by reference in its entirety). Myc is often referred to as a “master transcriptional regulator” with greater than 10,000 binding sites throughout the human genome, and Myc coordinates a transcriptional regulatory network that consists of approximately 15% of all genes (Dang (2013) Cold Spring Harbor Perspectives in Medicine 3: pii: a014217, which is incorporated by reference in its entirety). The Myc target gene network is extremely large and diverse. Myc specifically controls gene expression programs responsible for cell proliferation, growth, metabolism, and evasion from apoptosis.

The MYC gene is one of the most commonly dysregulated genes across all human cancers, and Myc expression often correlates with disease prognosis, metastatic potential, therapeutic resistance, and poor patient outcomes (Kalkat et al. (2017) Genes (Basel) 8: 151, which is incorporated by reference in its entirety). Myc deregulation can occur genetically, epigenetically, and post-transcriptionally through a wide variety of mechanisms. The widespread pleiotropic transcriptional changes induced by deregulated Myc act to potently transform cells to an oncogenic phenotype. Cancers with high levels of Myc have been experimentally shown to be correlated to its expression, such that inhibition of dysregulated Myc expression leads to rapid proliferative arrest and apoptosis of tumor cells (Felsher & Bishop (1999) Molecular Cell 4:199-207).

In some embodiments, a Myc inhibitor (e.g., a dominant negative variant of Myc) is or comprises a variant of a leucine zipper domain of a Myc polypeptide. In some embodiments, a Myc inhibitor (e.g., a dominant negative variant of Myc) comprises a variant of a basic helix-loop-helix DNA-binding domain of a Myc polypeptide. In some embodiments, a Myc inhibitor (e.g., a dominant negative variant of Myc) lacks a transactivation domain of a Myc polypeptide.

In some embodiments, a Myc inhibitor (e.g., a dominant negative variant of Myc) dimerizes with a Myc polypeptide (e.g., a wild-type Myc polypeptide), e.g., to inhibit Myc from dimerizing with its obligate heterodimerization partner Max.

In some embodiments, a Myc inhibitor (e.g., a dominant negative variant of Myc) dimerizes with a Max polypeptide (e.g., a wild-type Max polypeptide), e.g., to inhibit Myc from dimerizing with its obligate heterodimerization partner Max. In some embodiments, a dimer formed between a Myc inhibitor (e.g., a dominant negative variant of Myc) and Max can bind to an E-box sequence to form a complex that does not promote transcription.

In some embodiments, a Myc inhibitor (e.g., a dominant negative variant of Myc) does not interfere with Myc/Miz-1 dimerization and/or transcriptional repression.

In some embodiments, a Myc inhibitor (e.g., a dominant negative variant of Myc) is or comprises an OmoMYC polypeptide. An OmoMYC polypeptide is a mutant polypeptide derived from the basic helix-loop-helix and leucine zipper regions of a Myc polypeptide (Soucek et al. (1998) Oncogene 17: 1202199, which is incorporated by reference in its entirety). The mutations confer four amino acid substitutions within the Myc leucine zipper. As the leucine zipper comprises the interface driving heterodimerization, these substitutions alter the protein's binding affinity for other leucine zipper-containing proteins. An OmoMYC polypeptide is able to dimerize with wild type Myc. The affinity of Myc/OmoMYC heterodimers for E-box sequences is greatly reduced compared to Myc/Max heterodimers. Thus, an OmoMYC polypeptide sequesters Myc into nonfunctional complexes, preventing Myc from dimerizing with Max, from binding to DNA at E-boxes, and from driving gene expression, effectively acting as a dominant negative. Furthermore, an OmoMYC polypeptide is able to dimerize with Max and bind to E-box sequences. However, because an OmoMYC polypeptide lacks the Myc transactivation domain, these complexes are nonfunctional, thereby acting as a competitive inhibitor of Myc/Max heterodimers for binding to target genes. In some embodiments, an OmoMYC polypeptide may enhance the repressive functions of Myc, for example by not interfering with Myc/Miz-1 dimerization and transcriptional repression (Savino et al. (2011) PLoS ONE 6: e22284, which is incorporated by reference in its entirety). In some embodiments, an OmoMYC polypeptide acts as an edge-specific perturbation of a Myc network, selectively inhibiting Myc-mediated transcriptional activation while promoting Myc-mediated transcriptional repression. In some embodiments, an OmoMYC polypeptide modulates a Myc transcriptome, e.g., modulating Myc activity towards repression, and/or switches Myc from a pro-oncogenic to a tumor suppressive role. In some embodiments, an OmoMYC polypeptide enhances Myc-induced apoptosis in a manner dependent on Myc expression level, representing a powerful therapeutic strategy that specifically affects only Myc-deregulated cells (Soucek et al. (2002) Cancer Research 62: 3507-10, which is incorporated by reference in its entirety).

In some embodiments, a Myc inhibitor (e.g., a dominant negative variant of Myc) is or comprises an amino acid sequence that is at least 70%, 75%, 80%, 85%, 90%, 91%, 92%, 93%, 94%, 95%, 96%, 97%, 98%, or 100% identical to the sequence of SEQ ID NO.: 3, which is set forth below:

(SEQ ID NO: 3)
MTEENVKRRTHNVLERQRRNELKRSFFALRDQIPELENNEKAPKVVILKK
ATAYILSVQAETQKLISEIDLLRKQNEQLKHKLEQLRNSCA

In some embodiments, a Myc inhibitor (e.g., a dominant negative variant of Myc) is or comprises an amino acid sequence that is based on the sequence of SEQ ID NO: 3 and includes 0-10 (e.g., 0, 1, 2, 3, 4, 5, 6, 7, 8, 9, 10) amino acid modifications to the sequence of SEQ ID NO: 3. Examples of amino acid modifications include, e.g., but not limited to replacement of amino acid side chains, substitution of amino acid residues, deletion of amino acid residues, and insertion of amino acid residues. In some embodiments, amino acid modification(s) are made to the sequence of SEQ ID NO: 3 such that the resulting Myc inhibitor (e.g., a dominant negative variant of Myc) retains at least 70% or more (including, e.g., at least 80%, at least 90%, at least 95%, at least 98% or more) of the activity (e.g., reducing or inhibiting expression and/or activity of Myc itself or its interaction with heterodimerization partners), as compared to a Myc inhibitor based on the sequence of SEQ ID NO: 3 without amino acid modifications. In some embodiments, a Myc inhibitor (e.g., a dominant negative variant of Myc) is or comprises the amino acid sequence of SEQ ID NO.: 3.

### II. Compositions

Other aspects of the present disclosure provides compositions comprising any component, or combination of components, of a nucleic acid expression system as described herein. In some embodiments, the compositions described herein are useful for improving delivery of RNA oligonucleotides (e.g., mRNA oligonucleotides) comprising a payload sequence. In some embodiments, the compositions described herein are useful for improving the effectiveness of RNA oligonucleotide therapeutics and vaccines. In some embodiments, the compositions described herein are useful for reducing non-specific toxicity induced by RNA oligonucleotide therapeutics and vaccines. In some embodiments, the compositions described herein are useful for reducing innate immunity-triggered suppression of protein translation and/or mRNA degradation. In some embodiments, the compositions described herein are useful for enhancing expression and/or activity of a payload sequence to be introduced into target cells.

In some embodiments, a composition comprises at least one RNA oligonucleotide comprising a payload sequence as described herein. In some embodiments, a composition comprises 1, 2, 3, 4, 5, 6, 7, 8, 9, or 10 RNA oligonucleotides, each comprising a payload sequence. In some embodiments of the compositions described herein, the payload sequence encodes a dominant negative variant of a Myc polypeptide as described herein.

In some embodiments, a composition comprises or further comprises least one (e.g., 1, 2, 3, 4, 5, 6, 7, 8, 9, or 10) RNA oligonucleotide comprising a sequence that encodes a US11 polypeptide (e.g., one as described herein). In some embodiments, a composition comprising an RNA oligonucleotide that encodes a US11 polypeptide may further comprise at least one oligonucleotide (e.g., at least one, at least two, at least three, at least four, or more oligonucleotides) encoding a helper polypeptide. In some such embodiments, such a helper polypeptide is or comprises a nuclear localization signal (NLS), a DNA mimic polypeptide, a viral modulator of innate immunity, a synthetic cell surface receptor, or combinations thereof. Examples of such helper polypeptides that can be used in compositions described herein include but are not limited to ones described in the International Patent Publication No. WO 2019/060631, the contents of which are incorporated herein by reference for the sole purposes described herein.

In some embodiments, a composition comprises at least one RNA oligonucleotide comprising a payload sequence and at least one RNA oligonucleotide comprising a sequence that encodes a US11 polypeptide. In some embodiments, a composition comprises at least one (e.g., 1, 2, 3, 4, 5, or more) RNA oligonucleotide comprising a payload sequence (e.g., encoding a Myc inhibitor as described herein) and at least one (e.g., 1, 2, 3, 4, 5, or more) RNA oligonucleotide comprising a sequence that encodes a US11 polypeptide (e.g., ones described herein).

In some embodiments, a composition comprises any one of the embodiments of a nucleic acid expression system described herein.

RNA oligonucleotides (e.g., comprising a payload sequence and/or encoding a US11 polypeptide) in any of nucleic acid expression systems and/or compositions described herein may be delivered as naked RNA oligonucleotides or complexed with a complexing agent, e.g., for protecting RNA oligonucleotides from degradation, and/or for facilitating cell delivery. Exemplary complexing agents include, but are not limited to lipids, polymers, or small arginine-rich peptide such as protamine. In some embodiments, RNA oligonucleotides (e.g., comprising a payload sequence or encoding a US11 polypeptide) in any of nucleic acid expression systems and/or compositions described herein may be encapsulated, e.g., in liposomes or other suitable carriers.

In some embodiments, any of compositions described herein can be a pharmaceutical composition.

Pharmaceutical Compositions

In some embodiments, an RNA oligonucleotide comprising a payload sequence that encodes a Myc inhibitor (e.g., a dominant negative variant of a Myc polypeptide, e.g., ones described herein) can be included in a pharmaceutical composition. In some embodiments, an RNA oligonucleotide comprising a sequence that encodes a US11 polypeptide (e.g., ones described herein) can be included in a pharmaceutical composition. In some embodiments, a pharmaceutical composition can comprise (i) an RNA oligonucleotide comprising a payload sequence (e.g., a payload sequence encoding a Myc inhibitor, e.g., ones described herein) and (ii) an RNA oligonucleotide comprising a sequence that encodes a US11 polypeptide (e.g., ones described herein).

In some embodiments, a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration is typically a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of a pharmaceutical composition described herein. For example, a unit dose of a pharmaceutical composition comprises a predetermined amount of at least one RNA oligonucleotide comprising a payload sequence (e.g., encoding a Myc inhibitor such as ones as described herein) and/or at least one RNA oligonucleotide comprising a sequence that encodes a US11 polypeptide (e.g., ones as described herein).

Relative amounts of any components in pharmaceutical compositions described herein, e.g., an RNA oligonucleotide comprising a payload sequence (e.g., encoding a Myc inhibitor such as ones as described herein), at least one RNA oligonucleotide comprising a sequence that encodes a US11 polypeptide (e.g., ones as described herein), a pharmaceutically acceptable excipient, and/or any additional ingredients can vary, depending upon the subject to be treated, target cells, and may also further depend upon the route by which the composition is to be administered.

Kits

Another aspect of the present disclosure further provides a pharmaceutical pack or kit comprising one or more containers filled with at least one RNA oligonucleotide comprising a payload sequence (e.g., encoding a Myc inhibitor such as ones as described herein) and/or at least one RNA oligonucleotide comprising a sequence that encodes a US11 polypeptide (e.g., ones as described herein). Kits may be used in any applicable method, including, for example, cell treatment, therapeutically or diagnostically. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Cells

Cells comprising any embodiment of a nucleic acid expression system described herein are also provided herein. For example, in some embodiments, a cell comprises an RNA oligonucleotide comprising a payload sequence (e.g., encoding a Myc inhibitor such as ones described herein) and an RNA oligonucleotide comprising a sequence that encodes a US11 polypeptide (e.g., ones described herein). In some embodiments, a payload sequence introduced into cells via an RNA oligonucleotide comprising a payload sequence that encodes a Myc inhibitor (e.g., ones described herein). In some embodiments, a US11 polypeptide introduced into cells via an RNA oligonucleotide is obtained or derived from a HSV polypeptide such as a HSV-1 polypeptide. In some embodiments, a US11 polypeptide introduced into cells via an RNA oligonucleotide is a US11 polypeptide (e.g., ones described herein).

Any cells can be chosen to express a payload sequence delivered via an RNA oligonucleotide. In some embodiments, cells to be contacted with any of compositions or nucleic acid expression systems described herein can be wild-type cells, normal cells, diseased cells (e.g., cancer cells), or transgenic cells. In some embodiments, cells to be contacted with any of compositions or nucleic acid expression systems described herein can be eukaryotic cells (e.g., mammalian cells).

In some embodiments, cells as provided herein are cells that have been previously treated at least once or more (e.g., 1, 2, 3, 4, 5, 6, 7, 8, 9, 10 times or more) with one or more oligonucleotides. In some embodiments, oligonucleotides that are previously introduced into cells are DNA oligonucleotides. In some embodiments, oligonucleotides that are previously introduced into cells are RNA oligonucleotides (e.g., mRNA oligonucleotides).

In some embodiments, cells as provided herein are cancer cells. For example, cancer cells may be from leukemia, neuroblastoma, lymphoma, breast cancer, colon cancer, lung cancer, ovarian cancer, thymoma, germ cell tumor, myeloma, melanoma, rectal cancer, stomach cancer, pancreatic cancer, testicular cancer, skin cancer, sarcoma, or brain cancer.

### IV. Methods of Uses

The present disclosure recognizes that challenges associated with cell treatment based on RNA oligonucleotides involve high immunogenicity associated with foreign RNA oligonucleotides to be introduced into cells. The present disclosure, among other things, also recognizes that while using non-standard base chemistries may reduce immunogenicity of mRNA therapeutics, such modification may adversely affect efficiencies of translating mRNA to corresponding peptides or polypeptides in cells. Further, concerns with residual immune response that precludes repeated dosing and/or high-level dosing still remain. Therefore, there remains a need in the field for methods of delivering to target cells RNA oligonucleotides that minimize activation of myriad innate immune sensors while are still efficiently recognized by translational machinery.

The present disclosure, at least in part, addresses this need and provides methods by which innate immunity-triggered suppression of protein translation, mRNA degradation, and non-specific toxicity induced by RNA oligonucleotides are reduced, thereby enhancing expression of RNA oligonucleotides in cells. Further, higher doses and/or repeated doses of RNA oligonucleotides can be applied to cells using any of methods described herein to improve or sustain expression of RNA oligonucleotides without adversely inducing non-specific cell toxicity that would otherwise generally induced by any RNA oligonucleotides. These advantages can be beneficial for delivering and improving the effectiveness of RNA therapeutics and vaccines.

Accordingly, methods for using any embodiment of nucleic acid expression systems, compositions, and pharmaceutical compositions described herein are provided. In some embodiments, a method comprises (i) contacting a target cell with an RNA oligonucleotide comprising a payload sequence (e.g., encoding a Myc inhibitor); and (ii) contacting the target cell with an RNA oligonucleotide comprising a sequence that encodes a US11 polypeptide.

RNA oligonucleotides encoding any payload sequences and/or any US11 polypeptides disclosed herein may be used in any embodiment of methods described herein. For example, a US11 polypeptide may comprise the sequence of SEQ ID No: 1 or 2. In some embodiments, a payload sequence may encode a Myc inhibitor (e.g., a dominant negative variant of a Myc polypeptide) including, e.g., ones described herein.

In some embodiments, an RNA oligonucleotide comprising a payload sequence used in methods described herein is a synthetic RNA oligonucleotide. In some embodiments, an RNA oligonucleotide comprising a payload sequence used in methods described herein is a mRNA oligonucleotide (e.g., a synthetic mRNA oligonucleotide).

In some embodiments, an RNA oligonucleotide comprising a sequence that encodes a US11 polypeptide used in methods described herein is a synthetic RNA oligonucleotide. In some embodiments, an RNA oligonucleotide comprising a sequence that encodes a US11 polypeptide used in methods described herein is a mRNA oligonucleotide (e.g., a synthetic mRNA oligonucleotide).

In some embodiments, methods described herein are for enhancing expression and/or activity of a payload sequence in a target cell when the payload sequence is introduced into the target cell in the presence of an RNA oligonucleotide (e.g., a mRNA oligonucleotide) encoding a US11 polypeptide. In some embodiments, expression and/or activity of a payload sequence in a target cell is enhanced by at least 30% or more, including, e.g., at least 40%, at least 50%, at least 60%, at least 70%, at least 80%, at least 90%, at least 95%, or more, as compared to expression and/or activity of the same payload sequence in the target cell in the absence of an RNA oligonucleotide comprising a sequence that encodes a US11 polypeptide. In some embodiments, expression and/or activity of a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to expression and/or activity of the same payload sequence in the target cell in the absence of an RNA oligonucleotide comprising a sequence that encodes a US11 polypeptide. Accordingly, in some embodiments, provided herein is a method for enhancing expression and/or activity of a payload sequence delivered via an RNA oligonucleotide, wherein the method comprises (a) contacting a target cell with an RNA oligonucleotide comprising a payload sequence (e.g., encoding a Myc inhibitor); and (b) contacting the target cell with an RNA oligonucleotide comprising a sequence that encodes a US11 polypeptide (e.g., ones described herein).

In some embodiments, methods described herein are for enhancing viability of a target cell upon contacting with an RNA oligonucleotide comprising a payload sequence and an RNA oligonucleotide (e.g., a mRNA oligonucleotide) encoding a US11 polypeptide. In some embodiments, viability of a target cell upon contacting with an RNA oligonucleotide comprising a payload sequence and an RNA oligonucleotide (e.g., a mRNA oligonucleotide) encoding a US11 polypeptide is enhanced by at least 30% or more, including, e.g., at least 40%, at least 50%, at least 60%, at least 70%, at least 80%, at least 90%, at least 95%, or more, as compared to viability of a target cell upon contacting with an RNA oligonucleotide comprising the same payload sequence in the absence of an RNA oligonucleotide comprising a sequence that encodes a US11 polypeptide. In some embodiments, viability of a target cell upon contacting with an RNA oligonucleotide comprising a payload sequence and an RNA oligonucleotide (e.g., a mRNA oligonucleotide) encoding a US11 polypeptide is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to viability of a target cell upon contacting with an RNA oligonucleotide comprising the same payload sequence in the absence of an RNA oligonucleotide comprising a sequence that encodes a US11 polypeptide. Accordingly, in some embodiments, provided herein is a method for enhancing viability of a target cell upon introduction of a payload sequence via an RNA oligonucleotide, wherein the method comprises (a) contacting a target cell with an RNA oligonucleotide comprising a payload sequence (e.g., encoding a Myc inhibitor); and (b) contacting the target cell with an RNA oligonucleotide comprising a sequence that encodes a US11 polypeptide (e.g., ones described herein).

In some embodiments, methods described herein are for reducing non-specific toxicity induced in a target cell by introduction of an RNA oligonucleotide comprising a payload sequence when the payload sequence is introduced into the target cell in the presence of an RNA oligonucleotide (e.g., a mRNA oligonucleotide) encoding a US11 polypeptide. In some embodiments, non-specific toxicity induced in a target cell by introduction of an RNA oligonucleotide comprising a payload sequence is reduced by at least 30% or more, including, e.g., at least 40%, at least 50%, at least 60%, at least 70%, at least 80%, at least 90%, at least 95%, at least 98%, at least 99% or more, as compared to non-specific toxicity induced in a target cell by an RNA oligonucleotide comprising the same payload sequence in the absence of an RNA oligonucleotide comprising a sequence that encodes a US11 polypeptide. Accordingly, in some embodiments, provided herein is a method for reducing non-specific cell toxicity induced by introduction of a payload sequence via an RNA oligonucleotide, wherein the method comprises (a) contacting a target cell with an RNA oligonucleotide comprising a payload sequence (e.g., encoding a Myc inhibitor); and (b) contacting the target cell with an RNA oligonucleotide comprising a sequence that encodes a US11 polypeptide (e.g., ones described herein).

In some embodiments, methods described herein are for reducing innate immunity-triggered suppression of protein translation when a payload sequence is introduced into a target cell in the presence of an RNA oligonucleotide (e.g., a mRNA oligonucleotide) encoding a US11 polypeptide. In some embodiments, innate immunity-triggered suppression of protein translation of an introduced payload sequence is reduced by at least 30% or more, including, e.g., at least 40%, at least 50%, at least 60%, at least 70%, at least 80%, at least 90%, at least 95%, at least 98%, at least 99% or more, as compared to innate immunity-triggered suppression of protein translation of an introduced payload sequence in the absence of an RNA oligonucleotide comprising a sequence that encodes a US11 polypeptide. Accordingly, in some embodiments, provided herein is a method for reducing innate immunity-triggered suppression of translating an introduced mRNA payload oligonucleotide into a corresponding payload peptide or polypeptide, wherein the method comprises (a) contacting a target cell with an RNA oligonucleotide comprising a payload sequence (e.g., encoding a Myc inhibitor); and (b) contacting the target cell with an RNA oligonucleotide comprising a sequence that encodes a US11 polypeptide (e.g., ones described herein).

In some embodiments, methods described herein are for reducing innate immunity-triggered mRNA degradation when a payload sequence encoded by a mRNA oligonucleotide is introduced into a target cell in the presence of an RNA oligonucleotide (e.g., a mRNA oligonucleotide) encoding a US11 polypeptide. In some embodiments, innate immunity-triggered degradation of an introduced mRNA payload oligonucleotide is reduced by at least 30% or more, including, e.g., at least 40%, at least 50%, at least 60%, at least 70%, at least 80%, at least 90%, at least 95%, at least 98%, at least 99% or more, as compared to innate immunity-triggered degradation of an introduced mRNA payload oligonucleotide in the absence of an RNA oligonucleotide comprising a sequence that encodes a US11 polypeptide. Accordingly, in some embodiments, provided herein is a method for reducing innate immunity-triggered degradation of an introduced mRNA payload oligonucleotide, wherein the method comprises (a) contacting a target cell with an RNA oligonucleotide comprising a payload sequence (e.g., encoding a Myc inhibitor); and (b) contacting the target cell with an RNA oligonucleotide comprising a sequence that encodes a US11 polypeptide (e.g., ones described herein).

Methods described herein can be used for in vitro, ex vivo and in vivo applications. Thus, cells to which RNA oligonucleotides (e.g., an RNA oligonucleotide comprising a payload sequence and/or an RNA oligonucleotide comprising a sequence that encodes a US11 polypeptide) are delivered can be, for example, cells cultured in vitro or ex vivo, cells within a tissue, or cells present in a subject or organism. In some embodiments, cells to which RNA oligonucleotides (e.g., an RNA oligonucleotide comprising a payload sequence and/or an RNA oligonucleotide comprising a sequence that encodes a US11 polypeptide) are delivered can be cells that have been previously treated at least once or more (e.g., 1, 2, 3, 4, 5, 6, 7, 8, 9, 10 times or more) with one or more oligonucleotides. In some embodiments, oligonucleotides that are previously introduced into cells can be DNA oligonucleotides. In some embodiments, oligonucleotides that are previously introduced into cells can be RNA oligonucleotides (e.g., mRNA oligonucleotides).

RNA oligonucleotides (e.g., an RNA oligonucleotide comprising a payload sequence and/or an RNA oligonucleotide comprising a sequence that encodes a US11 polypeptide) used in any methods described herein can be delivered to cells by known methods in the art, including, but not limited to, transfection into cells (e.g., via electroporation, chemical methods, etc.), delivery via particles (e.g., nanoparticles or liposomes), and/or administration to an organism (e.g., by any suitable administration route).

In some embodiments, cells subjected to a method described herein are present in a subject. Therefore, in these embodiments, a target cell present in a subject is contacted with an RNA oligonucleotide comprising a payload sequence (e.g., encoding a Myc inhibitor such as ones described herein) by administering the RNA oligonucleotide comprising the payload sequence to the subject. In some embodiments, a target cell present in a subject is contacted with an RNA oligonucleotide comprising a sequence that encodes a US11 polypeptide (e.g., ones described herein) by administering the RNA oligonucleotide the US11 polypeptide to the subject.

In some embodiments, methods, nucleic acid expression systems, and compositions described herein can be used for delivering an RNA oligonucleotide to a target cell for a gene therapy or RNA oligonucleotide therapy in a subject. In some embodiments, a target cell to be subjected to a method, nucleic acid expression system, and/or composition described herein is isolated from a subject. In some embodiments, a target cell can be autologous to a subject (i.e., from a subject). In some embodiments, a target cell can be non-autologous (i.e., allogeneic or xenogenic) to a subject.

In some embodiments, a target cell (e.g., for in vitro, ex vivo, or in vivo applications described herein) is contacted with an RNA oligonucleotide comprising a payload sequence and an RNA oligonucleotide comprising a sequence that encodes a US11 polypeptide concurrently. In some embodiments, a target cell (e.g., for in vitro, ex vivo, or in vivo applications described herein) is contacted with an RNA oligonucleotide comprising a payload sequence and an RNA oligonucleotide comprising a sequence that encodes a US11 polypeptide separately. For example, in some embodiments, an RNA oligonucleotide comprising a sequence that encodes a US11 polypeptide is delivered to a target cell, and an RNA oligonucleotide comprising a payload sequence is delivered to the target cell at a later time. In some embodiments, an RNA oligonucleotide comprising a payload sequence is delivered to a target cell during a time when innate immunity pathway is attenuated (e.g., temporarily attenuated by at least 10% or more including, e.g., at least 20%, at least 30%, at least 40%, or more) by an RNA oligonucleotide comprising a sequence that encodes a US11 polypeptide. For example, in some embodiments, an RNA oligonucleotide comprising a payload sequence is delivered to a target cell 30 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 1 week, 2 weeks, 3 weeks, 4 weeks, 6 weeks, or 8 weeks after an RNA oligonucleotide comprising a sequence that encodes a US11 polypeptide is delivered.

In some embodiments, a composition comprising at least one RNA oligonucleotide sequence that encodes a US11 polypeptide is delivered to a target cell that has been contacted with an RNA oligonucleotide comprising a payload sequence, such that the target cell receives both.

In some embodiments, a composition comprising an RNA oligonucleotide comprising a payload sequence is administered to a target cell that has been contacted with at least one RNA oligonucleotide sequence that encodes a US11 polypeptide, such that the target cell receives both.

Exemplary Therapeutic Uses: Cancer

As described above, nucleic acid expression systems, compositions (e.g., pharmaceutical compositions), and/or methods according to any embodiment of the present disclosure achieve surprising advantages relative to existing RNA therapy or gene therapy. For example, nucleic acid expression systems, compositions (e.g., pharmaceutical compositions), and/or methods involving an RNA oligonucleotide comprising a sequence that encodes a US11 polypeptide can reduce non-specific toxicity induced by an RNA oligonucleotide comprising a payload sequence that encodes a Myc inhibitor in a target cell, and/or enhancing expression and/or activity of a Myc inhibitor encoded by an RNA oligonucleotide, etc. As demonstrated in Example 2 herein, a nucleic acid expression system, a composition (e.g., a pharmaceutical composition), and/or a method according to some embodiments of the present disclosure effectively attenuated or killed cancer cells (e.g., Kras cancer cells) by contacting cancer cells with an RNA oligonucleotide comprising a payload sequence that encodes a Myc inhibitor (e.g., a dominant negative variant of a Myc polypeptide) in the presence of an RNA oligonucleotide comprising a sequence that encodes a US11 polypeptide (e.g., ones described herein). More importantly, such therapeutic effects (e.g., attenuating or reducing growth of, or killing cancer cells) were shown to be specifically induced by function and/or activity of a Myc inhibitor encoded by an RNA oligonucleotide, rather than contribution from non-specific toxicity associated with the immunogenicity of RNA oligonucleotides.

Accordingly, in some embodiments, a nucleic acid expression system, composition, and/or method described herein can be used for attenuating a cancer cell. For example, in some embodiments, a method for attenuating a cancer cell comprises (i) contacting a cancer cell with an RNA oligonucleotide comprising a payload sequence that encodes a Myc inhibitor; and (ii) contacting the cancer cell with an RNA oligonucleotide comprising a sequence that encodes a US11 polypeptide.

Any US11 polypeptide herein can be used to attenuate a cancer cell in a method described herein. For example, in some embodiments, a US11 polypeptide encoded by an RNA oligonucleotide is obtained or derived from a HSV or HSV-1 polypeptide) or a US11 polypeptide. In some embodiments, a US11 polypeptide or portion thereof encoded by an RNA oligonucleotide comprises the sequence of SEQ ID NO: 1 or SEQ ID NO: 2.

Any Myc inhibitor described herein can be used to attenuate a cancer cell in a method described herein. For example, in some embodiments, a Myc inhibitor encoded by a payload sequence to be delivered by an RNA oligonucleotide is a dominant negative variant of a Myc polypeptide, e.g., ones described herein.

In some embodiments, a Myc inhibitor encoded by a payload sequence to be delivered by an RNA oligonucleotide is OmoMYC. Introduction of OmoMYC into Myc-expressing cells inhibits Myc-driven transcriptional activation, as shown by decrease in expression of canonical Myc target genes (Fukazawa et al. (2010) Anticancer Research 30: 4193-200, which is incorporated by reference in its entirety). In some instances, OmoMYC expression may reverse Myc-induced transformation, inhibit cellular proliferation, reduce cellular viability, and/or promote apoptosis in a Myc-dependent cancer cell. In some instances where a cancer cell is present in vivo, OmoMYC expression may inhibit tumor formation, lead to regression of both early-stage and established tumors, inhibit tumor cell proliferation, induce tumor cell apoptosis and senescence, and/or extend overall survival in a subject having a Myc-expressing cancer cell. It was also previously shown that in a KRAS-driven murine lung cancer model, OmoMYC completely prevented the formation of lung tumors and completely cured mice harboring established lung tumors, with no evidence of relapse or resistance (Soucek et al. (2008) Nature 455: 679, which is incorporated by reference in its entirety). Thus, OmoMYC can represent a potent Myc inhibitor and cancer therapeutic. It is noted that systemic Myc inhibition can exert profound effects on healthy tissue, notably rapidly regenerating tissues including epidermis, testis, gastrointestinal tract, and bone marrow. However, these effects are well tolerated and can be rapidly and completely reversible in animals or mice, indicating an evident therapeutic window for OmoMYC treatment of Myc-expressing tumors.

In some embodiments, a cancer cell receiving an RNA oligonucleotide comprising a payload sequence that encodes a Myc inhibitor (e.g., a dominant negative variant of a Myc polypeptide such as OmoMYC) is a cancer cell expressing Myc. In some embodiments, expression and/or activity of a Myc polypeptide in a Myc-expressing cancer cell is at least 30% or more (including, e.g., at least 40%, at least 50%, at least 60%, at least 70%, at least 80%, at least 90%, at least 95%, or more) than that in a non-cancerous cell. In some embodiments, expression and/or activity of a Myc polypeptide in a Myc-expressing cancer cell is at least 1.1-fold or more (including, e.g., at least 1.5-fold, at least 2-fold, at least 3-fold, at least 4-fold, at least 5-fold, or more) than that in a non-cancerous cell.

In some embodiments, a cancer cell is attenuated after contact with an RNA oligonucleotide comprising a payload sequence encoding a Myc inhibitor and an RNA oligonucleotide comprising a sequence that encodes a US11 polypeptide, by reducing proliferation of a cancer cell. For example, in some embodiments, proliferation of a cancer cell subjected to a method described herein is reduced by at least 30% or more, including, e.g., at least 40%, at least 50%, at least 60%, at least 70%, at least 80%, at least 90%, or more, as compared to proliferation of a cancer cells not subjected to a method described herein.

In some embodiments, a cancer cell is attenuated after contact with an RNA oligonucleotide comprising a payload sequence encoding a Myc inhibitor and an RNA oligonucleotide comprising a sequence that encodes a US11 polypeptide, by inducing apoptosis of a cancer cell. For example, in some embodiments, apoptosis of a cancer cell subjected to a method described herein is induced by at least 30% or more, including, e.g., at least 40%, at least 50%, at least 60%, at least 70%, at least 80%, at least 90%, or more, as compared to apoptosis of a cancer cell not subjected to a method described herein.

In some embodiments, expression and/or activity of a payload sequence encoding a Myc inhibitor in a cancer cell is enhanced by at least 30% or more, including, e.g., at least 40%, at least 50%, at least 60%, at least 70%, at least 80%, at least 90%, at least 95%, or more, as compared to expression and/or activity of the same payload sequence in a cancer cell in the absence of an RNA oligonucleotide comprising a sequence that encodes a US11 polypeptide. In some embodiments, expression and/or activity of a payload sequence encoding a Myc inhibitor in a cancer cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to expression and/or activity of the same payload sequence in a cancer cell in the absence of an RNA oligonucleotide comprising a sequence that encodes a US11 polypeptide.

In some embodiments, non-specific toxicity induced in a cancer cell by introduction of an RNA oligonucleotide comprising a payload sequence is reduced by at least 30% or more, including, e.g., at least 40%, at least 50%, at least 60%, at least 70%, at least 80%, at least 90%, at least 95%, at least 98%, at least 99% or more, as compared to non-specific toxicity induced in a cancer cell by an RNA oligonucleotide comprising the same payload sequence in the absence of an RNA oligonucleotide comprising a sequence that encodes a US11 polypeptide.

In some embodiments, a cancer cell to be treated by a method described herein is from leukemia, neuroblastoma, lymphoma, breast cancer, colon cancer, lung cancer, ovarian cancer, thymoma, germ cell tumor, myeloma, melanoma, rectal cancer, stomach cancer, pancreatic cancer, testicular cancer, skin cancer, sarcoma, or brain cancer.

The following embodiments as described below are also within the scope of the disclosure:
1. A nucleic acid expression system comprising:
   (i) an RNA oligonucleotide comprising a payload sequence that encodes a Myc inhibitor, and
   (ii) an RNA oligonucleotide comprising a sequence that encodes a US11 polypeptide.
2. The nucleic acid expression system of embodiment 1, wherein the RNA oligonucleotide of (i) is a synthetic RNA oligonucleotide.
3. The nucleic acid expression system of embodiment 1 or 2, wherein the RNA oligonucleotide of (ii) is a synthetic RNA oligonucleotide.
4. The nucleic acid expression system of any one of embodiments 1-3, wherein the RNA oligonucleotide of (i) is a messenger RNA (mRNA) oligonucleotide.
5. The nucleic acid expression system of any one of embodiments 1-4, wherein the RNA oligonucleotide of (ii) is a mRNA oligonucleotide.
6. The nucleic acid expression system of any one of embodiments 1-5, wherein the US11 polypeptide is or includes an RNA binding domain of a US11 polypeptide.
7. The nucleic acid expression system of any one of embodiments 1-6, wherein the US11 polypeptide comprises the sequence of SEQ ID NO.: 1 or SEQ ID NO: 2.
8. The nucleic acid expression system of any one of embodiments 1-7, wherein the Myc inhibitor reduces expression and/or activity of Myc.
9. The nucleic acid expression system of any one of embodiments 1-8, wherein the Myc inhibitor comprises a dominant negative variant of a Myc polypeptide.
10. The nucleic acid expression system of any one of embodiments 1-9, wherein the Myc inhibitor is or comprises a variant of at least one domain of a Myc polypeptide, the at least one domain being selected from the group consisting of a basic helix-loop-helix DNA-binding domain, a leucine zipper domain, and a transactivation domain of a Myc polypeptide.
11. The nucleic acid expression system of any one of embodiments any one of embodiments 1-10, wherein the Myc inhibitor is or comprises a variant of a leucine zipper domain of a Myc polypeptide.
12. The nucleic acid expression system of embodiment 11, wherein the Myc inhibitor includes a basic helix-loop-helix DNA-binding domain of a Myc polypeptide.
13. The nucleic acid expression system of embodiment 11 or 12, wherein the Myc inhibitor lacks a transactivation domain of a Myc polypeptide.
14. The nucleic acid expression system of any one of embodiments 1-13, wherein the Myc inhibitor dimerizes with a Myc polypeptide.
15. The nucleic acid expression system of any one of embodiments 1-14, wherein the Myc inhibitor dimerizes with a Max polypeptide to form a dimer.
16. The nucleic acid expression system of embodiment 15, wherein the dimer binds to an E-box sequence to form a complex that does not promote transcription.
17. The nucleic acid expression system of any one of embodiments 1-16, wherein the Myc inhibitor does not interfere with Myc/Miz-1 dimerization and/or transcriptional repression.
18. The nucleic acid expression system of any one of embodiments 1-17, wherein the Myc inhibitor is or comprises an OmoMYC polypeptide.
19. The nucleic acid expression system of any one of embodiments 1-18, wherein the Myc inhibitor is or comprises an amino acid sequence that is at least 90%, 91%, 92%, 93%, 94%, 95%, 96%, 97%, 98%, or 100% identical to the sequence of SEQ ID NO.: 3.
20. The nucleic acid expression system of any one of embodiments 1-18, wherein the Myc inhibitor is or comprises an amino acid sequence that is based on the sequence of SEQ ID NO.: 3 and includes 0-10 amino acid modifications to the sequence of SEQ ID NO.: 3.
21. The nucleic acid expression system of any one of embodiments 1-18, wherein the Myc inhibitor is or comprises the amino acid sequence of SEQ ID NO.: 3.
22. A composition comprising the nucleic acid expression system of any one of embodiments 1-21.
23. The composition of embodiment 22, wherein the composition is a pharmaceutical composition.
24. The composition of embodiment 23, further comprising a pharmaceutically acceptable carrier.
25. A cell comprising the nucleic acid expression system of any one of embodiments 1-21.
26. The cell of embodiment 25, wherein the cell is a cancer cell.
27. A pharmaceutical composition comprising:
   (i) an RNA oligonucleotide comprising a payload sequence that encodes a dominant negative variant of a Myc polypeptide, and
   (ii) a pharmaceutically acceptable carrier.
28. The pharmaceutical composition of embodiment 27, wherein the dominant negative variant is or comprises a variant of at least one domain of a Myc polypeptide, the at least one domain being selected from a basic helix-loop-helix DNA-binding domain, a leucine zipper domain, and a transactivation domain of a Myc polypeptide.
29. The pharmaceutical composition of embodiment 27 or 28, wherein the dominant negative variant comprises a variant of a leucine zipper domain of a Myc polypeptide.
30. The pharmaceutical composition of embodiment 29, wherein the dominant negative variant includes a basic helix-loop-helix DNA-binding domain of a Myc polypeptide.
31. The pharmaceutical composition of embodiment 29 or 30, wherein the dominant negative lacks a transactivation domain of a Myc polypeptide.
32. The pharmaceutical composition of any one of embodiments 27-31, wherein the dominant negative variant dimerizes with a Myc polypeptide.
33. The pharmaceutical composition of any one of embodiments 27-32, wherein the dominant negative variant dimerizes with a Max polypeptide to form a dimer.
34. The pharmaceutical composition of embodiment 33, wherein the dimer binds to an E-box sequence to form a complex that does not activate transcription.
35. The pharmaceutical composition of any one of embodiments 27-34 wherein the dominant negative variant does not interfere with Myc/Miz-1 dimerization and/or transcriptional repression.
36. The pharmaceutical composition of any one of embodiments 27-35, wherein the dominant negative variant is or comprises an OmoMYC polypeptide.
37. The pharmaceutical composition of any one of embodiments 27-36, wherein the dominant negative variant is or comprises an amino acid sequence that is at least 90%, 91%, 92%, 93%, 94%, 95%, 96%, 97%, 98%, or 100% identical to the sequence of SEQ ID NO.: 3.
38. The pharmaceutical composition of any one of embodiments 27-36, wherein the dominant negative variant is or comprises an amino acid sequence that is based on the sequence of SEQ ID NO.: 3 and includes 0-10 amino acid modifications to the sequence of SEQ ID NO.: 3.
39. The pharmaceutical composition of any one of embodiments 27-36, wherein the dominant negative variant is or comprises the amino acid sequence of SEQ ID NO.: 3.
40. The pharmaceutical composition of any one of embodiments 27-39, wherein the RNA oligonucleotide comprising the payload sequence is a synthetic RNA oligonucleotide.
41. The pharmaceutical composition of any one of embodiments 27-40, wherein the RNA oligonucleotide comprising the payload sequence is a mRNA oligonucleotide.
42. A method comprising:
   a. contacting a target cell with an RNA oligonucleotide comprising a payload sequence that encodes a Myc inhibitor; and
   b. contacting the target cell with an RNA oligonucleotide comprising a sequence that encodes a US11 polypeptide.
43. The method of embodiment 42, wherein the RNA oligonucleotide comprising the payload sequence is a synthetic RNA oligonucleotide.
44. The method of embodiment 42 or 43, wherein the RNA oligonucleotide comprising the sequence that encodes the US11 polypeptide is a synthetic RNA oligonucleotide.
45. The method of any one of embodiments 42-44, wherein the RNA oligonucleotide comprising the payload sequence is a messenger RNA (mRNA) oligonucleotide.
46. The method of any one of embodiments 42-45, wherein the RNA oligonucleotide comprising the sequence that encodes the US11 polypeptide is a mRNA oligonucleotide.
47. The method of any one of embodiments 42-46, wherein the US11 polypeptide is or includes an RNA binding domain of a US11 polypeptide.
48. The method of any one of embodiments 42-47, wherein the US11 polypeptide comprises the sequence of SEQ ID NO.: 1 or SEQ ID NO: 2.
49. The method of any one of embodiments 42-48, wherein the method is for enhancing expression and/or activity of the payload sequence in the target cell.
50. The method of embodiment 49, wherein the expression and/or activity of the payload sequence in the target cell is enhanced by at least 30% or more, as compared to the expression and/or activity of the payload sequence in the target cell in the absence of the RNA oligonucleotide comprising the sequence that encodes the US11 polypeptide.
51. The method of any one of embodiments 42-50, wherein the method is for enhancing viability of the target cell upon contacting with the RNA oligonucleotide comprising the payload sequence and the RNA oligonucleotide comprising the sequence that encodes the US11 polypeptide.
52. The method of embodiment 51, wherein the viability of the target cell upon contacting with the RNA oligonucleotide comprising the payload sequence and the RNA oligonucleotide comprising the sequence that encodes the US11 polypeptide is enhanced by at least 30% or more, as compared to the viability of the target cell upon contacting with the RNA oligonucleotide comprising the payload sequence in the absence of the RNA oligonucleotide comprising the sequence that encodes the US11 polypeptide.
53. The method of any one of embodiments 42-52, wherein the method is for reducing non-specific toxicity induced in the target cell by the RNA oligonucleotide comprising the payload sequence.
54. The method of embodiment 53, wherein the non-specific toxicity induced in the target cell by the RNA oligonucleotide comprising the payload sequence is reduced by at least 30% or more, as compared to the non-specific toxicity induced in the target cell by the RNA oligonucleotide comprising the payload sequence in the absence of the RNA oligonucleotide comprising the sequence that encodes the US11 polypeptide.
55. The method of any one of embodiments 42-54, wherein the target cell is previously contacted at least once by one or more oligonucleotides.
56. The method of any one of embodiments 42-55, wherein the target cell is contacted with the RNA oligonucleotide comprising the payload sequence and the RNA oligonucleotide comprising the sequence that encodes the US11 polypeptide concurrently.
57. The method of any one of embodiments 42-55, wherein the target cell is contacted with the RNA oligonucleotide comprising the payload sequence and the RNA oligonucleotide comprising the sequence that encodes the US11 polypeptide separately.
58. The method of embodiment 57, wherein the target cell is contacted with the RNA oligonucleotide comprising the payload sequence and the RNA oligonucleotide comprising the sequence that encodes the US11 polypeptide separately within 24 hours or less.
59. The method of any one of embodiments 42-58, wherein the target cell is present in a subject.
60. The method of embodiment 59, wherein the target cell present in the subject is contacted with the RNA oligonucleotide comprising the payload sequence by administering the RNA oligonucleotide comprising the payload sequence to the subject.
61. The method of embodiment 59 or 60, wherein the target cell present in the subject is contacted with the RNA oligonucleotide comprising the sequence that encodes the US11 polypeptide by administering the RNA oligonucleotide comprising encoding the US11 polypeptide to the subject.
62. The method of any one of embodiments 42-61, wherein the target cell is a cancer cell.
63. A method of attenuating a cancer cell comprising:
   a. contacting a cancer cell with an RNA oligonucleotide comprising a payload sequence that encodes a Myc inhibitor; and
   b. contacting the cancer cell with an RNA oligonucleotide comprising a sequence that encodes a US11 polypeptide.
64. The method of embodiment 63, wherein the US11 polypeptide is or includes an RNA binding domain of a US11 polypeptide.
65. The method of embodiment 63 or 64, wherein the US11 polypeptide comprises the sequence of SEQ ID NO.: 1 or SEQ ID NO: 2.
66. The method of any one of embodiments 63-65, wherein expression and/or activity of the payload sequence in the cancer cell is enhanced by at least 30% or more, as compared to the expression and/or activity of the payload sequence in the cancer cell in the absence of the RNA oligonucleotide comprising the sequence that encodes the US11 polypeptide.
67. The method of any one of embodiments 63-66, wherein non-specific toxicity induced in the cancer cell by the RNA oligonucleotide comprising the payload sequence is reduced by at least 30% or more, as compared to the non-specific toxicity induced in the cancer cell by the RNA oligonucleotide comprising the payload sequence in the absence of the RNA oligonucleotide comprising the sequence that encodes the US11 polypeptide.
68. The method of any one of embodiments 63-67, wherein the cancer cell is previously contacted at least once by one or more oligonucleotides.
69. The method of any one of embodiments 63-68, wherein the Myc inhibitor reduces expression and/or activity of Myc.
70. The method of any one of embodiments 63-69, wherein the Myc inhibitor comprises a dominant negative variant of a Myc polypeptide.
71. The method of any one of embodiments 63-70, wherein the Myc inhibitor is or comprises a variant of at least one domain of a Myc polypeptide, the at least one domain being selected from a basic helix-loop-helix DNA-binding domain, a leucine zipper domain, and a transactivation domain of a Myc polypeptide.
72. The method of any one of embodiments 63-71, wherein the Myc inhibitor is or comprises a variant of a leucine zipper domain of a Myc polypeptide.
73. The method of embodiment 72, wherein the Myc inhibitor includes a basic helix-loop-helix DNA-binding domain of a Myc polypeptide.
74. The method of embodiment 72 or 73, wherein the Myc inhibitor lacks a transactivation domain of a Myc polypeptide.
75. The method of any one of embodiments 63-74, wherein the Myc inhibitor dimerizes with a Myc polypeptide.
76. The method of any one of embodiments 63-75, wherein the Myc inhibitor dimerizes with a Max polypeptide to form a dimer.
77. The method of embodiment 76, wherein the dimer binds to an E-box sequence to form a complex that does not promote transcription.
78. The method of any one of embodiments 63-77, wherein the Myc inhibitor does not interfere with Myc/Miz-1 dimerization and/or transcriptional repression.
79. The method of any one of embodiments 63-78, wherein the Myc inhibitor is or comprises an OmoMYC polypeptide.
80. The method of any one of embodiments 63-79, wherein the Myc inhibitor is or comprises an amino acid sequence that is at least 90%, 91%, 92%, 93%, 94%, 95%, 96%, 97%, 98%, or 100% identical to the sequence of SEQ ID NO.: 3.
81. The method of any one of embodiments 63-79, wherein the Myc inhibitor is or comprises an amino acid sequence that is based on the sequence of SEQ ID NO.: 3 and includes 0-10 amino acid modifications to the sequence of SEQ ID NO.: 3.
82. The method of any one of embodiments 63-79, wherein the Myc inhibitor is or comprises the amino acid sequence of SEQ ID NO.: 3.
83. The method of any one of embodiments 63-82, wherein the cancer cell is from leukemia, neuroblastoma, lymphoma, breast cancer, colon cancer, lung cancer, ovarian cancer, thymoma, germ cell tumor, myeloma, melanoma, rectal cancer, stomach cancer, pancreatic cancer, testicular cancer, skin cancer, sarcoma, or brain cancer.
84. The method of any one of embodiments 63-83, wherein the RNA oligonucleotide comprising the payload sequence is a synthetic RNA oligonucleotide.
85. The method of any one of embodiments 63-84, wherein the RNA oligonucleotide comprising the sequence that encodes the US11 polypeptide is a synthetic RNA oligonucleotide.
86. The method of any one of embodiments 63-85, wherein the RNA oligonucleotide comprising the payload sequence is a mRNA oligonucleotide.
87. The method of any one of embodiments 63-86, wherein the RNA oligonucleotide comprising the sequence that encodes the US11 polypeptide is a mRNA oligonucleotide.
88. The method of any one of embodiments 63-87, wherein the cancer cell is contacted with the RNA oligonucleotide comprising the payload sequence and the RNA oligonucleotide comprising the sequence that encodes the US11 polypeptide concurrently.
89. The method of any one of embodiments 63-87, wherein the cancer cell is contacted with the RNA oligonucleotide comprising the payload sequence and the RNA oligonucleotide comprising the sequence that encodes the US11 polypeptide separately.
90. The method of embodiment 89, wherein the cancer cell is contacted with the RNA oligonucleotide comprising the payload sequence and the RNA oligonucleotide comprising the sequence that encodes the US11 polypeptide separately within 24 hours or less.

Other features of the invention will become apparent in the course of the following descriptions of exemplary embodiments, which are given for illustration of the invention and are not intended to be limiting thereof.

### EXEMPLIFICATION

### Example 1—Transfection Efficiency of Co-Delivery of an RNA Oligonucleotide Comprising a Sequence that Encodes a Model Payload with an RNA Oligonucleotide Comprising a Sequence that Encodes a US11 Polypeptide

The present Example describes synthesis of an RNA oligonucleotide comprising a sequence that encodes an exemplary US11 polypeptide and an RNA oligonucleotide comprising a model payload sequence and further demonstrates that co-delivery of an RNA oligonucleotide comprising a model payload sequence with an RNA oligonucleotide comprising a sequence that encodes an exemplary US11 polypeptide can increase expression of the model payload. While this study assessed expression of a payload in target cells when an RNA oligonucleotide comprising a sequence that encodes a model payload (e.g., a model reporter polypeptide) was delivered to target cells following delivery of an RNA oligonucleotide comprising a sequence that encodes a US11 polypeptide, similar technical effects can also be observed when both an RNA polynucleotide comprising a model payload sequence and an RNA polynucleotide encoding a US11 polypeptide are delivered concurrently to the target cells (see Example 2).

Preparation of an RNA Oligonucleotide Comprising a Sequence that Encodes an Exemplary Model Payload Sequence

Firefly luciferase mRNA synthesis: The luc2 gene encoding an optimized version of firefly luciferase was amplified from pGL4.10[luc2] (Promega) with Luc2_fwd (shown below) and Luc2_rev (shown below) using Herculase II polymerase (Agilent) with an annealing temperature of 70° C. and with 250 mM betaine supplementation. The PCR product was cleaned up using DNA Clean & Concentrator-5 (Zymo Research) and digested with Dpn I (New England Biolabs). The digested PCR product as then amplified with T7-AGG_fwd and 120 pA_rev using Herculase II polymerase with an annealing temperature of 50° C. and with 1 M betaine supplementation. The secondary PCR product was cleaned up using DNA Clean & Concentrator-5 and used as a template for T7 transcription. Specifically, the HighScribe T7 High Yield (New England Biolabs) was used to set up reactions consisting of 40 ng/μL luc2 template, 10 mM of each nucleotide (ATP, GTP, UTP, CTP), 10 mM of CleanCap Reagent AG (Trilink Biotech), 1×T7 buffer, and 0.1 μL/μL T7 RNA polymerase mix. The mRNA transcription reactions were carried out for 2 hrs at 37° C. mRNA products were subsequently treated with TURBO DNase (Thermo Fisher) and purified using the MEGAclear Transcription Clean-Up Kit (Thermo Fisher), eluting into 0.1 mM EDTA pH 8. The yield was quantified using a NanoDrop spectrophotometer (Thermo Fisher). The purified products were then treated with 1×RNAsecure (Thermo Fisher) and heated to 60° C. in order to inactivate any contaminating RNAses. The integrity of the synthesized products were confirmed using 2% EX gels (Thermo Fisher).

The sequences of exemplary primers used are shown as follows:

Luc2_Avd:
(SEQ ID NO: 4)
CTTGTTCTTTTTGCAGAAGCTCAGAATAAACGCTCAACTTTGGCCACCat
ggaagatgccaaaaacattaagaagggc
Luc2_rev:
(SEQ ID NO: 5)
AGAATGTGAAGAAACTTTCTTTTTATTAGGAGCAGATACGAATGGCTACA
TTTTGGGGGACAACATTTTGTAAAGTGTAAGTTGGTATTATGTAGCTTAG
AGACTCCATTCGGGTGTTCTTGAGGCTGGTCTATCATTAcacggcgatct
tgccgcc
T7-ACG_Avd:
(SEQ ID NO: 6)
gaattTAATACGACTCACTATAAGGcttgttctttttgcagaagc
120pA_rev:
(SEQ ID NO: 7)
TTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTT
TTTTTTTTTTTTTTTTTTTTagaatgtgaagaaactttctttttattag

An exemplary sequence of a luc2 mRNA product is shown as follows:

(SEQ ID NO: 8)
AGGCUUGUUCUUUUUGCAGAAGCCUUGUUCUUUUUGCAGAAGCUCAGAAU
AAACGCUCAACUUUGGCCACCAUGGAAGAUGCCAAAAACAUUAAGAAGGG
CCCAGCGCCAUUCUACCCACUCGAAGACGGGACCGCCGGCGAGCAGCUGC
ACAAAGCCAUGAAGCGCUACGCCCUGGUGCCCGGCACCAUCGCCUUUACC
GACGCACAUAUCGAGGUGGACAUUACCUACGCCGAGUACUUCGAGAUGAG
CGUUCGGCUGGCAGAAGCUAUGAAGCGCUAUGGGCUGAAUACAAACCAUC
GGAUCGUGGUGUGCAGCGAGAAUAGCUUGCAGUUCUUCAUGCCCGUGUUG
GGUGCCCUGUUCAUCGGUGUGGCUGUGGCCCCAGCUAACGACAUCUACAA
CGAGCGCGAGCUGCUGAACAGCAUGGGCAUCAGCCAGCCCACCGUCGUAU
UCGUGAGCAAGAAAGGGCUGCAAAAGAUCCUCAACGUGCAAAAGAAGCUA
CCGAUCAUACAAAAGAUCAUCAUCAUGGAUAGCAAGACCGACUACCAGGG
CUUCCAAAGCAUGUACACCUUCGUGACUUCCCAUUUGCCACCCGGCUUCA
ACGAGUACGACUUCGUGCCCGAGAGCUUCGACCGGGACAAAACCAUCGCC
CUGAUCAUGAACAGUAGUGGCAGUACCGGAUUGCCCAAGGGCGUAGCCCU
ACCGCACCGCACCGCUUGUGUCCGAUUCAGUCAUGCCCGCGACCCCAUCU
UCGGCAACCAGAUCAUCCCCGACACCGCUAUCCUCAGCGUGGUGCCAUUU
CACCACGGCUUCGGCAUGUUCACCACGCUGGGCUACUUGAUCUGCGGCUU
UCGGGUCGUGCUCAUGUACCGCUUCGAGGAGGAGCUAUUCUUGCGCAGCU
UGCAAGACUAUAAGAUUCAAUCUGCCCUGCUGGUGCCCACACUAUUUAGC
UUCUUCGCUAAGAGCACUCUCAUCGACAAGUACGACCUAAGCAACUUGCA
CGAGAUCGCCAGCGGCGGGGCGCCGCUCAGCAAGGAGGUAGGUGAGGCCG
UGGCCAAACGCUUCCACCUACCAGGCAUCCGCCAGGGCUACGGCCUGACA
GAAACAACCAGCGCCAUUCUGAUCACCCCCGAAGGGGACGACAAGCCUGG
CGCAGUAGGCAAGGUGGUGCCCUUCUUCGAGGCUAAGGUGGUGGACUUGG
ACACCGGUAAGACACUGGGUGUGAACCAGCGCGGCGAGCUGUGCGUCCGU
GGCCCCAUGAUCAUGAGCGGCUACGUUAACAACCCCGAGGCUACAAACGC
UCUCAUCGACAAGGACGGCUGGCUGCACAGCGGCGACAUCGCCUACUGGG
ACGAGGACGAGCACUUCUUCAUCGUGGACCGGCUGAAGAGCCUGAUCAAA
UACAAGGGCUACCAGGUAGCCCCAGCCGAACUGGAGAGCAUCCUGCUGCA
ACACCCCAACAUCUUCGACGCCGGGGUCGCCGGCCUGCCCGACGACGAUG
CCGGCGAGCUGCCCGCCGCAGUCGUCGUGCUGGAACACGGUAAAACCAUG
ACCGAGAAGGAGAUCGUGGACUAUGUGGCCAGCCAGGUUACAACCGCCAA
GAAGCUGCGCGGUGGUGUUGUGUUCGUGGACGAGGUGCCUAAAGGACUGA
CCGGCAAGUUGGACGCCCGCAAGAUCCGCGAGAUUCUCAUUAAGGCCAAG
AAGGGCGGCAAGAUCGCCGUGUAAUGAUAGACCAGCCUCAAGAACACCCG
AAUGGAGUCUCUAAGCUACAUAAUACCAACUUACACUUUACAAAAUGUUG
UCCCCCAAAAUGUAGCCAUUCGUAUCUGCUCCUAAUAAAAAGAAAGUUUC
UUCACAUUCUAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAA

Preparation of an RNA Oligonucleotide Comprising a Sequence that Encodes an Exemplary US11 Polypeptide

US11 mRNA synthesis: The US11 gene containing 5′ and 3′ UTRs was synthesized, for example, as a gBlock (Integrated DNA Technologies) and amplified, e.g., with T7-AGG_fwd and 120 pA_rev using a polymerase (e.g., Herculase II polymerase) with an annealing temperature of 50° C. and with 1 M betaine supplementation. The PCR product was cleaned up, e.g., using DNA Clean & Concentrator-5, and used as a template for T7 transcription. The transcription reactions were carried out, e.g., using HighScribe T7 High Yield as described above for luc2 mRNA synthesis, except using 20 ng/μL US11 template. mRNA products were DNAse digested as described for luc2 mRNA synthesis. Purification was carried out, e.g., using Dynabeads Oligo (dT)25 (Thermo Fisher). For example, the 0.2375 μL of the magnetic beads were added per μL of T7 synthesis reaction in Binding Buffer (1×RNAsecure, 1M LiCl, 2 mM EDTA, 20 mM Tris-Cl pH 7.5). The mixture was heated to 60° C. to denature the mRNA, cooled on ice, and incubated at room temperature for 5 min. The mRNA-bound beads were then washed using Wash Buffer (1×RNAsecure, 150 mM LiCl, 1 mM EDTA, 10 mM Tris-Cl pH 7.5). Bead-bound mRNA was eluted into Elution Buffer (1×RNAsecure, 1 mM EDTA, 10 mM Tris-Cl pH 7.5) by heating to 80° C. for 2 min. The yield was quantified using the QuantiFlour RNA System (Promega). The integrity of the synthesized products were confirmed using 1% EX gels.

An exemplary sequence of the US11 gBlock is shown as follows:

(SEQ ID NO: 9)
CTTGTTCTTTTTGCAGAAGCTCAGAATAAACGCTCAACTTTGGCCACCat
gagccagacccaacccccggccccagttgggccgggcgacccagatgttt
acttaaaaggcgtgccgtccgccggcatgcaccccagaggtgttcacgca
cctcgaggacacccgcgcatgatctccggacccccgcaacggggtgataa
cgatcaagcggcggggcaatgtggagattcgggtctactacgagtcggtg
cggacactacgatctcgaagccatctgaagccgtccgaccgccaacaatc
cccaggacaccgcgtgttccccgggagccccgggttccgcgaccaccccg
agaacctagggaacccagagtaccgcgagctcccagagaccccagggtac
cgcgtgaccccagggatccacgacaaccGcgTtcCccAagggagccccgg
tctcccCgTgaAccccggtctcccagggagccccggaccccacgcacccc
ccgcgaaccacgtacggctcgcggTtctgtaTAATGATAGACCAGCCTCA
AGAACACCCGAATGGAGTCTCTAAGCTACATAATACCAACTTACACTTTA
CAAAATGTTGTCCCCCAAAATGTAGCCATTCGTATCTGCTCCTAATAAAA
AGAAAGTTTCTTCACATTCT

An exemplary sequence of a US11 mRNA product is shown as follows:

(SEQ ID NO: 10)
AGGCUUGUUCUUUUUGCAGAAGCCUUGUUCUUUUUGCAGAAGCUCAGAAU
AAACGCUCAACUUUGGCCACCAUGAGCCAGACCCAACCCCCGGCCCCAGU
UGGGCCGGGCGACCCAGAUGUUUACUUAAAAGGCGUGCCGUCCGCCGGCA
UGCACCCCAGAGGUGUUCACGCACCUCGAGGACACCCGCGCAUGAUCUCC
GGACCCCCGCAACGGGGUGAUAACGAUCAAGCGGCGGGGCAAUGUGGAGA
UUCGGGUCUACUACGAGUCGGUGCGGACACUACGAUCUCGAAGCCAUCUG
AAGCCGUCCGACCGCCAACAAUCCCCAGGACACCGCGUGUUCCCCGGGAG
CCCCGGGUUCCGCGACCACCCCGAGAACCUAGGGAACCCAGAGUACCGCG
AGCUCCCAGAGACCCCAGGGUACCGCGUGACCCCAGGGAUCCACGACAAC
CGCGUUCCCCAAGGGAGCCCCGGUCUCCCCGUGAACCCCGGUCUCCCAGG
GAGCCCCGGACCCCACGCACCCCCCGCGAACCACGUACGGCUCGCGGUUC
UGUAUAAUGAUAGACCAGCCUCAAGAACACCCGAAUGGAGUCUCUAAGCU
ACAUAAUACCAACUUACACUUUACAAAAUGUUGUCCCCCAAAAUGUAGCC
AUUCGUAUCUGCUCCUAAUAAAAAGAAAGUUUCUUCACAUUCUAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAA

Exemplary Treatment of Target Cells with an RNA Oligonucleotide Comprising a Model Payload Sequence and an RNA Oligonucleotide Comprising a Sequence that Encodes a US11 Polypeptide

A549 MAVS-transfection: Mitochondrial antiviral signaling (MAVS) knock-out cancer cells such as lung cancer cells (e.g., A549-Dual KO-MAVS cells (InvivoGen)) were cultured, e.g., in high glucose GlutaMAX Dulbecco's Modified Eagle Medium (Thermo Fisher) supplemented with 10% heat-inactivated fetal bovine serum, 100 units/mL penicillin, 100 μg/mL streptomycin, 10 μg/mL blasticidin, and 100 μg/mL zeocin and maintained at 37° C. and 5% CO2. Cells were plated in a 96-well plate at 4,000 cells/well in antibiotic-free culture media one day prior to treatment (e.g., transfection). Variable amounts of a mRNA oligonucleotide comprising a sequence that encodes a US11 polypeptide was delivered to cells per well, e.g., via transfections, e.g., using Lipofectamine MessengerMAX at a 1.5 uL reagent per μg mRNA. An appropriate amount of a mRNA oligonucleotide comprising a sequence that encodes a model payload, e.g., a reporter polypeptide (e.g., 300 ng of a mRNA oligonucleotide comprising a sequence that encodes luc2) was then delivered to the cells, e.g., by transfections, following delivery of the mRNA oligonucleotide comprising a sequence that encodes a US11 polypeptide. For example, a mRNA oligonucleotide comprising a sequence that encodes a model payload, e.g., luc2, was delivered to the cells, e.g., 1 day following delivery of the mRNA oligonucleotide comprising a sequence that encodes a US11 polypeptide. Levels of a model payload (e.g., luciferase levels) were assayed, e.g., 1 day following a payload (e.g., luc2) transfection, for example, using the ONE-Glo+Tox Luciferase Reporter and Cell Viability assay (Promega).

### Results

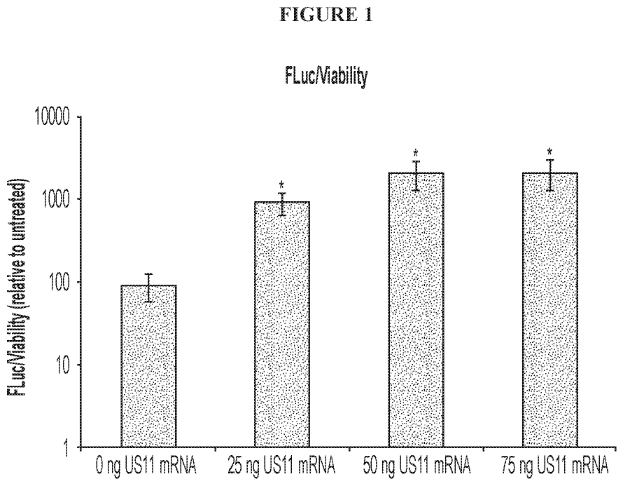

As shown in FIG. 1, expression of a model payload (e.g., luciferase) in the target cells (e.g., cancer cells) was increased by co-delivery of a mRNA oligonucleotide comprising a model payload sequence (e.g., luc2) with a mRNA oligonucleotide comprising a sequence that encodes a US11 polypeptide. For example, a significant (p<0.05, n=2-3 replicate mRNA preparations and transfections) improvement in firefly luciferase expression was observed in the cells with co-expression of US11.

### Example 2—Effects of Co-Delivery of an RNA Oligonucleotide Comprising a Sequence that Encodes a Model Myc Inhibitor with an RNA Oligonucleotide Comprising a Sequence that Encodes a US11 Polypeptide on Target Cells

The present Example demonstrates that co-delivery of an RNA oligonucleotide comprising a model payload sequence to target cells with an RNA oligonucleotide comprising a sequence that encodes an exemplary US11 polypeptide can reduce non-specific toxicity induced in the target cells by the RNA oligonucleotide comprising a model payload sequence. The present Example further demonstrates that co-delivery of an RNA oligonucleotide comprising a model payload sequence to target cells with an RNA oligonucleotide comprising a sequence that encodes an exemplary US11 polypeptide can improve viability of the target cells upon delivery of the RNA oligonucleotide comprising the model payload sequence into the target cells. While this study assessed non-specific toxicity and cell viability when both an RNA oligonucleotide comprising a sequence that encodes a model payload (e.g., a model Myc inhibitor) and an RNA oligonucleotide comprising a sequence that encodes a US11 polypeptide was concurrently delivered to target cells, similar technical effects can be exerted on the target cells when an RNA oligonucleotide comprising a sequence that encodes a model payload is delivered to target cells following delivery of an RNA oligonucleotide comprising a sequence that encodes a US11 polypeptide. See Example 1. Further, this Example demonstrates that similar technical effects were exerted on cells that have been previously treated (e.g., transfected) at least one or more (e.g., once, twice, or three times) with one or more oligonucleotides (e.g., RNA oligonucleotides encoding a payload).

In addition, the present Example demonstrates the effectiveness of attenuating cancer cells by co-delivery of an RNA oligonucleotide comprising a sequence that encodes a model Myc inhibitor (e.g., a dominant negative variant of a Myc polypeptide) to cancer cells with an RNA oligonucleotide comprising a sequence that encodes an exemplary US11 polypeptide.

Preparation of an RNA Oligonucleotide Comprising a Sequence that Encodes an Exemplary US11 Polypeptide

US11 mRNA synthesis: The US11 gBlock (e.g., as described in Example 1) was cloned into pCR II-Blunt-TOPO using the Zero Blunt TOPO PCR Cloning Kit (Thermo Fisher). T7 template was then generated by amplifying the cloned US11 gene with T7-AGG_fwd and 120 pA_rev using Herculase II polymerase with an annealing temperature of 50° C. The template was purified up using DNA Clean & Concentrator-25 (Zymo Research), digested with Dpn I, further purified with DNA Clean & Concentrator-5, and treated with 1×RNAsecure. The transcription reactions were carried out using HighScribe T7 High Yield, e.g., as described in Example 1. mRNA products were DNAse digested, purified, and characterized, e.g., as described for luc2 mRNA synthesis in Example 1.

Preparation of an RNA Oligonucleotide Comprising a Sequence that Encodes an Exemplary Myc Inhibitor

OmoMYC mRNA synthesis: The OmoMYC gene and two negative controls consisting of the codon-scrambled Omomyc sequence were synthesized as gBlocks. The constructs were cloned into cloned, amplified, and synthesized into mRNA as described for US11 mRNA synthesis above.

An exemplary sequence of a synthesized OmoMYC gBlock is shown as follows:

(SEQ ID NO: 11)
taaCTTGTTCTTTTTGCAGAAGCTCAGAATAAACGCTCAACTTTGGCCAC
Catgaccgaggagaatgtcaagaggcgaacacacaacgtcttggagcgcc
agaggaggaacgagctaaaacggagcttttttgccctgcgtgaccagatc
ccggagttggaaaacaatgaaaaggcccccaaggtagttatccttaaaaa
agccacagcatacatcctgtccgtccaagcagagacgcaaaagctcattt
ctgaaatcgacttgttgcggaaacaaaacgaacagttgaaacacaaactt
gaacagctacggaactcttgtgcgTAATGATAGACCAGCCTCAAGAACAC
CCGAATGGAGTCTCTAAGCTACATAATACCAACTTACACTTTACAAAATG
TTGTCCCCCAAAATGTAGCCATTCGTATCTGCTCCTAATAAAAAGAAAGT
TTCTTCACATTCT

An exemplary sequence of a synthesized scramble control 1 gBlock is shown as follows:

(SEQ ID NO: 12)
taaCTTGTTCTTTTTGCAGAAGCTCAGAATAAACGCTCAACTTTGGCCAC
Catgctagtcaacctccttaaggacgagcgtcgggcgaacatccgcgcca
aacggcacttgacactgcaggtccagatcaatcggcaacaagccgtcaag
gaaacctctgtagcattgaggacggagttgcacaacctgaggctaatcac
attgaataaagaaccgcaatttgttaaagccaaaaaccgatcctttatcg
acagggagcaggagtctaaattgtgtgaaaacgcaaagtaccagagcgag
cttcccaagattaaagaagaggaaTAATGATAGACCAGCCTCAAGAACAC
CCGAATGGAGTCTCTAAGCTACATAATACCAACTTACACTTTACAAAATG
TTGTCCCCCAAAATGTAGCCATTCGTATCTGCTCCTAATAAAAAGAAAGT
TTCTTCACATTCT

An exemplary sequence of a synthesized scramble control 2 gBlock is shown as follows:

(SEQ ID NO: 13)
taaCTTGTTCTTTTTGCAGAAGCTCAGAATAAACGCTCAACTTTGGCCAC
Catgaacaagaactctatccggcaatctcttaaaaacgttcggttggacg
aagtcgcaaacgcacacttgcaacagagggaagtcaaaccgatcgacatc
aagcgcctgagcaaagccgagaacaaatttcaatacttgagggtcgaaaa
gcagacactattggccgagcccaaagagtgtacggagcgtaataaggagt
ttcaggtagaactgaggacacgagagcagcgggcccacttgattacccta
cttctcgaaatcaaagcgaattccTAATGATAGACCAGCCTCAAGAACAC
CCGAATGGAGTCTCTAAGCTACATAATACCAACTTACACTTTACAAAATG
TTGTCCCCCAAAATGTAGCCATTCGTATCTGCTCCTAATAAAAAGAAAGT
TTCTTCACATTCT

An exemplary sequence of a mRNA product of OmoMYC is shown as follows:

(SEQ ID NO: 14)
AGGCUUGUUCUUUUUGCAGAAGCCUUGUUCUUUUUGCAGAAGCUCAGAAU
AAACGCUCAACUUUGGCCACCAUGACCGAGGAGAAUGUCAAGAGGCGAAC
ACACAACGUCUUGGAGCGCCAGAGGAGGAACGAGCUAAAACGGAGCUUUU
UUGCCCUGCGUGACCAGAUCCCGGAGUUGGAAAACAAUGAAAAGGCCCCC
AAGGUAGUUAUCCUUAAAAAAGCCACAGCAUACAUCCUGUCCGUCCAAGC
AGAGACGCAAAAGCUCAUUUCUGAAAUCGACUUGUUGCGGAAACAAAACG
AACAGUUGAAACACAAACUUGAACAGCUACGGAACUCUUGUGCGUAAUGA
UAGACCAGCCUCAAGAACACCCGAAUGGAGUCUCUAAGCUACAUAAUACC
AACUUACACUUUACAAAAUGUUGUCCCCCAAAAUGUAGCCAUUCGUAUCU
GCUCCUAAUAAAAAGAAAGUUUCUUCACAUUCUAAAAAAA

An exemplary sequence of a mRNA product of scramble control 1 is shown as follows:

(SEQ ID NO: 15)
AGGCUUGUUCUUUUUGCAGAAGCCUUGUUCUUUUUGCAGAAGCUCAGAAU
AAACGCUCAACUUUGGCCACCAUGCUAGUCAACCUCCUUAAGGACGAGCG
UCGGGCGAACAUCCGCGCCAAACGGCACUUGACACUGCAGGUCCAGAUCA
AUCGGCAACAAGCCGUCAAGGAAACCUCUGUAGCAUUGAGGACGGAGUUG
CACAACCUGAGGCUAAUCACAUUGAAUAAAGAACCGCAAUUUGUUAAAGC
CAAAAACCGAUCCUUUAUCGACAGGGAGCAGGAGUCUAAAUUGUGUGAAA
ACGCAAAGUACCAGAGCGAGCUUCCCAAGAUUAAAGAAGAGGAAUAAUGA
UAGACCAGCCUCAAGAACACCCGAAUGGAGUCUCUAAGCUACAUAAUACC
AACUUACACUUUACAAAAUGUUGUCCCCCAAAAUGUAGCCAUUCGUAUCU
GCUCCUAAUAAAAAGAAAGUUUCUUCACAUUCUAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAA

An exemplary sequence of a mRNA product of scramble control 2 is shown as follows:

(SEQ ID NO: 16)
AGGCUUGUUCUUUUUGCAGAAGCCUUGUUCUUUUUGCAGAAGCUCAGAAU
AAACGCUCAACUUUGGCCACCAUGAACAAGAACUCUAUCCGGCAAUCUCU
UAAAAACGUUCGGUUGGACGAAGUCGCAAACGCACACUUGCAACAGAGGG
AAGUCAAACCGAUCGACAUCAAGCGCCUGAGCAAAGCCGAGAACAAAUUU
CAAUACUUGAGGGUCGAAAAGCAGACACUAUUGGCCGAGCCCAAAGAGUG
UACGGAGCGUAAUAAGGAGUUUCAGGUAGAACUGAGGACACGAGAGCAGC
GGGCCCACUUGAUUACCCUACUUCUCGAAAUCAAAGCGAAUUCCUAAUGA
UAGACCAGCCUCAAGAACACCCGAAUGGAGUCUCUAAGCUACAUAAUACC
AACUUACACUUUACAAAAUGUUGUCCCCCAAAAUGUAGCCAUUCGUAUCU
GCUCCUAAUAAAAAGAAAGUUUCUUCACAUUCUAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAA

Exemplary Treatment of Target Cells with an RNA Oligonucleotide Comprising a Model Payload Sequence and an RNA Oligonucleotide Comprising a Sequence that Encodes a US11 Polypeptide

A549 MAVS-transfection: Mitochondrial antiviral signaling (MAVS) knock-out cancer cells such as lung cancer cells (e.g., A549-Dual KO-MAVS cells (InvivoGen)) were cultured, e.g., as described in Example 1. The cells were plated in a 24-well plate at 25,000 cells/well in culture media one day prior to transfection. The cells were treated (e.g., by transfection) with either (i) a mRNA oligonucleotide comprising a sequence that encodes a Myc inhibitor (e.g., 250 ng mRNA oligonucleotide comprising a sequence that encodes a dominant negative variant of a Myc polypeptide or portions thereof such as OmoMYC), or (ii) a mixture of a mRNA oligonucleotide comprising a sequence that encodes a Myc inhibitor (e.g., 175 ng mRNA oligonucleotide comprising a sequence that encodes a dominant negative variant of a Myc polypeptide or portions thereof such as OmoMYC) and a mRNA oligonucleotide comprising a sequence that encodes a US11 polypeptide (e.g., 75 ng mRNA oligonucleotide comprising a sequence that encodes a US11 polypeptide). An exemplary mixture comprised 250 ng total mRNA oligonucleotides with 25% US11 spike-in. The mRNA oligonucleotides were delivered to cancer cells, e.g., by transfection. For example, transfections were carried out, e.g., using Lipofectamine MessengerMAX at a 1.5 μL reagent per μg mRNA. On day 5 following delivery (e.g., transfection), cells were collected, e.g., using TrypLE Express (Thermo Fisher). Live cells were counted, e.g., using a Countess II Automated Cell Counter (Thermo Fisher) and EVE Cell Counting Slides (NanoEnTek), and a portion of the collected cells (e.g., 25% of the collected cells) were plated to a fresh 24-well plate. The plated cells were treated again (e.g., by transfection), e.g., on day 1 following plating, and were passaged and counted, e.g., on day 6 following plating. The process was repeated for multiple treatments (e.g., a total of at least 3 repeated transfections).

### Results

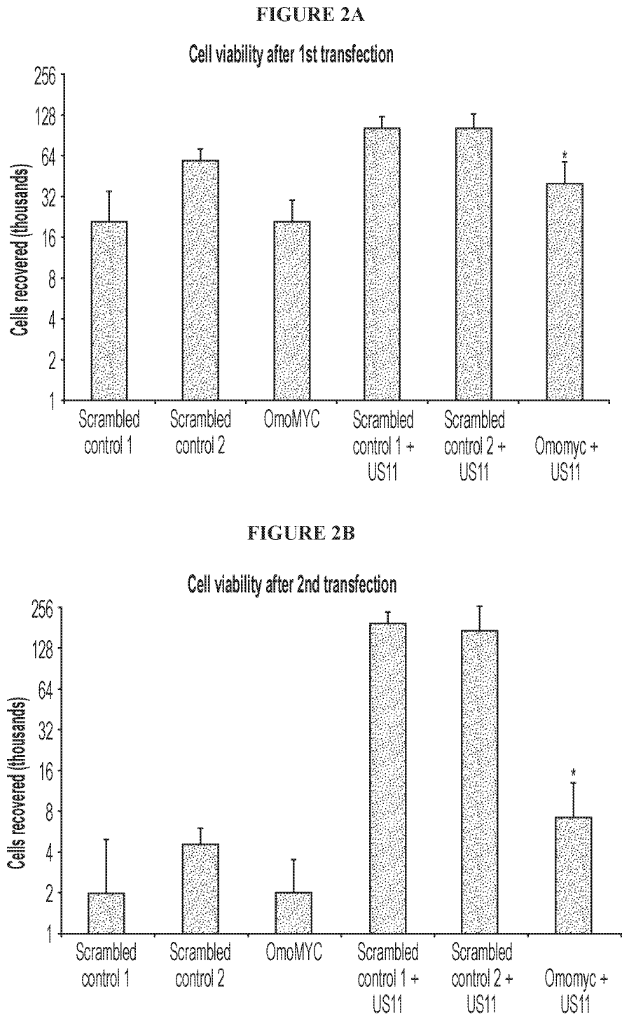

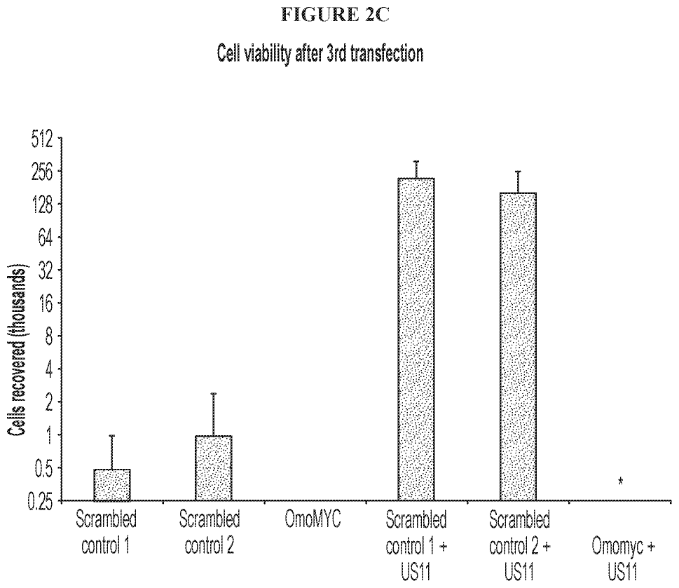

As shown in FIGS. 2A-2C, delivery of mRNA oligonucleotides encoding either a payload (e.g., a Myc inhibitor such as OmoMYC) or a negative control (e.g., scrambled sequences) alone (e.g., in the absence of mRNA oligonucleotides encoding a US11 polypeptide) induced cell death and thus lowered cell viability upon treatment (e.g., transfection). This data indicate that delivery of mRNA oligonucleotides at a tested dose into target cells can induce non-specific toxicity. However, an equivalent total amount of mRNA oligonucleotides containing a 25% mRNA oligonucleotides encoding a US11 polypeptide resulted in selective toxicity with mRNA oligonucleotides encoding a payload (e.g., a Myc inhibitor such as OmoMYC), which indicates selective effect exerted by the payload (e.g., inhibition of the MYC/KRAS pathway by OmoMYC). Such technical effects were also observed in cells after repeated treatments (e.g., by transfections). It is noted that A549 cells used in this study have the G12S MYC mutation (Mahoney et al. (2009) British Journal of Cancer 100(2), p. 370, which is incorporated by reference in its entirety).

In addition, as shown in FIGS. 2A-2C, cancer cells treated by co-delivery of a mRNA oligonucleotide encoding a Myc inhibitor (e.g., OmoMYC) and a mRNA oligonucleotide encoding a US11 polypeptide had significantly lower viability (p<0.05, n=3 replicate mRNA preparations and transfections) than those treated by co-delivery of a mRNA oligonucleotide encoding a negative control (e.g., a scramble sequence) and a mRNA oligonucleotide encoding a US11 polypeptide. Such technical effects were also observed in cells after repeated treatments (e.g., by transfections).

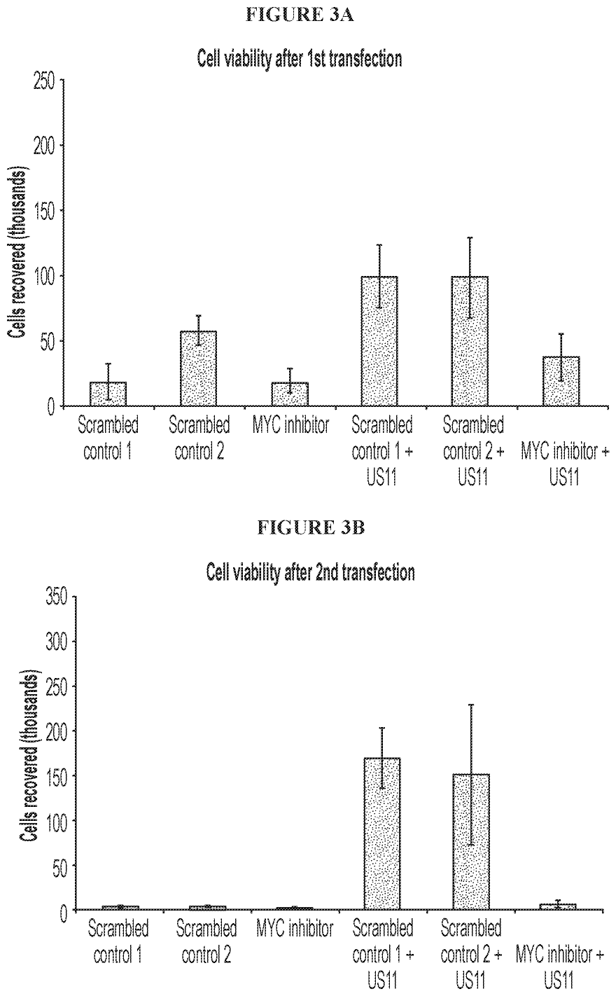

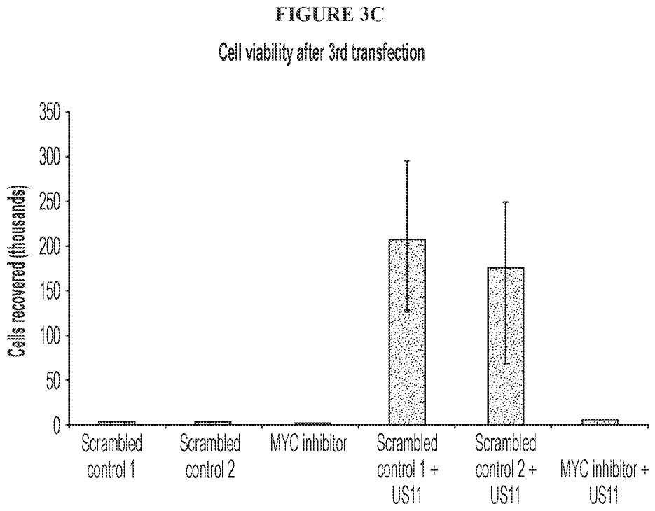

Similar studies as described in Example 2 were performed with an RNA oligonucleotide comprising a sequence that encodes a Myc inhibitor and an RNA oligonucleotide comprising a sequence that encodes a US11 polypeptide, according to another embodiment described herein. Similar to FIGS. 2A-2B, cancer cells treated by co-delivery of a mRNA oligonucleotide encoding a Myc inhibitor and a mRNA oligonucleotide encoding a US11 polypeptide had significantly lower viability than those treated by co-delivery of a mRNA oligonucleotide encoding a negative control (e.g., a scramble sequence) and a mRNA oligonucleotide encoding a US11 polypeptide. Such technical effects were also observed in cells after repeated treatments (e.g., by transfections). Results are shown in FIGS. 3A-3C.

These results indicate that delivery of a mRNA oligonucleotide comprising a sequence that encodes a Myc inhibitor (e.g., OmoMYC) to cancer cells in the presence of a mRNA oligonucleotide comprising a sequence that encodes a US11 polypeptide can help to extend the therapeutic window of a Myc inhibitor.

### Example 3—In Vitro and In Vivo Dose Response Studies

In vitro dose response studies, e.g., using different cancer cell lines such as wild type A549, other cancer cell lines, e.g., with mutated Kras, and negative controls, e.g., cancer cell lines with no mutations in the Kras/Myc pathway, are performed to determine the impacts of various amounts of an RNA oligonucleotide comprising a payload sequence (e.g., a Myc inhibitor) and an RNA oligonucleotide comprising a sequence that encodes a US11 polypeptide on, for example, expression of payloads in target cells, viability of cells upon delivery of the RNA oligonucleotides, and non-specific toxicity induced by the RNA oligonucleotides, e.g., as described in Example 2.

In vivo dose response studies, e.g., using tumor xenografts of various cancer cell lines, are performed to determine impacts of various amounts of an RNA oligonucleotide comprising a payload sequence (e.g., a Myc inhibitor) and an RNA oligonucleotide comprising a sequence that encodes a US11 polypeptide on, for example, expression of payloads in target cells, viability of cells upon delivery of the RNA oligonucleotides, and non-specific toxicity induced by the RNA oligonucleotides, e.g., as described in Example 2.

### Example 4—In Vitro Dose-Response and Toxicity Studies Using an RNA Oligonucleotide Comprising a Payload Sequence without an RNA Oligonucleotide Comprising a Sequence that Encodes a US11 Polypeptide

Similar in vitro dose response and toxicity studies, e.g., as described in Example 1, using an RNA oligonucleotide comprising a payload sequence (e.g., a payload sequence encoding a Myc inhibitor such as OmoMYC) without an RNA oligonucleotide comprising a sequence that encodes a US11 polypeptide, are performed to identify the therapeutic of OmoMYC when it is delivered in the absence of an RNA oligonucleotide comprising a US11 polypeptide.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A nucleic acid expression system comprising:
(i) an RNA oligonucleotide comprising a payload sequence that encodes a Myc inhibitor, and
(ii) an RNA oligonucleotide comprising a sequence that encodes a US11 polypeptide.

2. The nucleic acid expression system of claim 1, wherein the RNA oligonucleotide of (i) and/or the RNA oligonucleotide of (ii) is or comprises a synthetic RNA oligonucleotide.

3. The nucleic acid expression system of any one of claims 1-2, wherein the RNA oligonucleotide of (i) and/or the RNA oligonucleotide of (ii) is or comprises a messenger RNA (mRNA) oligonucleotide.

4. The nucleic acid expression system of any one of claims 1-3, wherein the US11 polypeptide is or includes an RNA binding domain of a US11 polypeptide.

5. The nucleic acid expression system of any one of claims 1-4, wherein the US11 polypeptide comprises the sequence of SEQ ID NO.: 1 or SEQ ID NO: 2.

6. The nucleic acid expression system of any one of claims 1-5, wherein the Myc inhibitor reduces expression and/or activity of Myc.

7. The nucleic acid expression system of any one of claims 1-6, wherein the Myc inhibitor is or comprises a dominant negative variant of a Myc polypeptide.

8. The nucleic acid expression system of any one of claims 1-7, wherein the Myc inhibitor is or comprises a variant of at least one domain of a Myc polypeptide, the at least one domain being selected from the group consisting of a basic helix-loop-helix DNA-binding domain, a leucine zipper domain, and a transactivation domain of a Myc polypeptide.

9. The nucleic acid expression system of any one of claims 1-8, wherein the Myc inhibitor includes one or more of the following characteristics:
(a) the Myc inhibitor is or comprises a variant of a leucine zipper domain of a Myc polypeptide;
(b) the Myc inhibitor is or comprises a variant of basic helix-loop-helix DNA binding domain of a Myc polypeptide; and
(c) the Myc inhibitor lacks a transactivation domain of a Myc polypeptide.

10. The nucleic acid expression system of any one of claims 1-9, wherein the Myc inhibitor dimerizes with a wild-type Myc polypeptide.

11. The nucleic acid expression system of any one of claims 1-10, wherein the Myc inhibitor dimerizes with a wild-type Max polypeptide to form a dimer.

12. The nucleic acid expression system of claim 11, wherein the dimer binds to an E-box sequence to form a complex that does not promote transcription.

13. The nucleic acid expression system of any one of claims 1-12, wherein the Myc inhibitor does not interfere with Myc/Miz-1 dimerization and/or transcriptional repression.

14. The nucleic acid expression system of any one of claims 1-13, wherein the Myc inhibitor is or comprises an OmoMYC polypeptide.

15. The nucleic acid expression system of any one of claims 1-14, wherein the Myc inhibitor is or comprises an amino acid sequence that is at least 90%, 91%, 92%, 93%, 94%, 95%, 96%, 97%, 98%, or 100% identical to the sequence of SEQ ID NO.: 3.

16. The nucleic acid expression system of any one of claims 1-15, wherein the Myc inhibitor is or comprises an amino acid sequence that is based on the sequence of SEQ ID NO.: 3 and includes 0-10 amino acid modifications to the sequence of SEQ ID NO.: 3.

17. The nucleic acid expression system of any one of claims 1-16, wherein the Myc inhibitor is or comprises the amino acid sequence of SEQ ID NO.: 3.

18. A pharmaceutical composition comprising:
(i) an RNA oligonucleotide comprising a payload sequence that encodes a dominant negative variant of a Myc polypeptide, and
(ii) a pharmaceutically acceptable carrier.

19. The pharmaceutical composition of claim 18, wherein the dominant negative variant is or comprises a variant of at least one domain of a Myc polypeptide, the at least one domain being selected from a basic helix-loop-helix DNA-binding domain, a leucine zipper domain, and a transactivation domain of a Myc polypeptide.

20. The pharmaceutical composition of claim 18 or 19, wherein the Myc inhibitor includes one or more of the following characteristics:
(a) the Myc inhibitor is or comprises a variant of a leucine zipper domain of a Myc polypeptide;
(b) the Myc inhibitor is or comprises a variant of basic helix-loop-helix DNA binding domain of a Myc polypeptide; and
(c) the Myc inhibitor lacks a transactivation domain of a Myc polypeptide.

21. The pharmaceutical composition of any one of claims 18-20, further comprising an RNA oligonucleotide comprising a sequence that encodes a US11 polypeptide.

22. A method comprising:
a. contacting a target cell with an RNA oligonucleotide comprising a payload sequence that encodes a Myc inhibitor; and
b. contacting the target cell with an RNA oligonucleotide comprising a sequence that encodes a US11 polypeptide.

23. The method of claim 22, wherein the method is for enhancing expression and/or activity of the payload sequence in the target cell.

24. The method of claim 23, wherein the expression and/or activity of the payload sequence in the target cell is enhanced by at least 30% or more, as compared to the expression and/or activity of the payload sequence in the target cell in the absence of the RNA oligonucleotide comprising the sequence that encodes the US11 polypeptide.

25. The method of any one of claims 22-24 wherein the method is for enhancing viability of the target cell upon contacting with the RNA oligonucleotide comprising the payload sequence and the RNA oligonucleotide comprising the sequence that encodes the US11 polypeptide.

26. The method of claim 25, wherein the viability of the target cell upon contacting with the RNA oligonucleotide comprising the payload sequence and the RNA oligonucleotide comprising the sequence that encodes the US11 polypeptide is enhanced by at least 30% or more, as compared to the viability of the target cell upon contacting with the RNA oligonucleotide comprising the payload sequence in the absence of the RNA oligonucleotide comprising the sequence that encodes the US11 polypeptide.

27. The method of any one of claims 22-26, wherein the method is for reducing non-specific toxicity induced in the target cell by the RNA oligonucleotide comprising the payload sequence.

28. The method of claim 27, wherein the non-specific toxicity induced in the target cell by the RNA oligonucleotide comprising the payload sequence is reduced by at least 30% or more, as compared to the non-specific toxicity induced in the target cell by the RNA oligonucleotide comprising the payload sequence in the absence of the RNA oligonucleotide comprising the sequence that encodes the US11 polypeptide.

29. The method of any one of claims 22-28, wherein the target cell is previously contacted at least once by one or more oligonucleotides.

30. The method of any one of claims 22-29, wherein the target cell is contacted with the RNA oligonucleotide comprising the payload sequence and the RNA oligonucleotide comprising the sequence that encodes the US11 polypeptide concurrently.

31. The method of any one of claims 22-29, wherein the target cell is contacted with the RNA oligonucleotide comprising the payload sequence and the RNA oligonucleotide comprising the sequence that encodes the US11 polypeptide separately.

32. The method of claim 31, wherein the target cell is contacted with the RNA oligonucleotide comprising the payload sequence and the RNA oligonucleotide comprising the sequence that encodes the US11 polypeptide separately within 24 hours or less.

33. The method of any one of claims 22-32, wherein the target cell is present in a subject.

34. The method of claim 33, wherein the target cell present in the subject is contacted with the RNA oligonucleotide comprising the payload sequence by administering the RNA oligonucleotide comprising the payload sequence to the subject.

35. The method of claim 33 or 34, wherein the target cell present in the subject is contacted with the RNA oligonucleotide comprising the sequence that encodes the US11 polypeptide by administering the RNA oligonucleotide comprising encoding the US11 polypeptide to the subject.

36. The method of any one of claims 22-35, wherein the target cell is a cancer cell.

37. A method of attenuating a cancer cell comprising:
a. contacting a cancer cell with an RNA oligonucleotide comprising a payload sequence that encodes a Myc inhibitor; and
b. contacting the cancer cell with an RNA oligonucleotide comprising a sequence that encodes a US11 polypeptide.

38. The method of claim 37, wherein non-specific toxicity induced in the cancer cell by the RNA oligonucleotide comprising the payload sequence is reduced by at least 30% or more, as compared to the non-specific toxicity induced in the cancer cell by the RNA oligonucleotide comprising the payload sequence in the absence of the RNA oligonucleotide comprising the sequence that encodes the US11 polypeptide.

39. The method of claim 37 or 38, wherein the cancer cell is from leukemia, neuroblastoma, lymphoma, breast cancer, colon cancer, lung cancer, ovarian cancer, thymoma, germ cell tumor, myeloma, melanoma, rectal cancer, stomach cancer, pancreatic cancer, testicular cancer, skin cancer, sarcoma, or brain cancer.


SEQUENCE LISTING (17 sequences)

SEQ ID NO: 1 - protein, 161 aa, Herpes simplex virus type 1
MSQTQPPAPVGPGDPDVYLKGVPSAGMHPRGVHAPRGHPRMISGPPQRGDNDQAAGQCGDSGLLRVGADTTISKPSEAVRPPTIPRTPRVPREPRVPRPPREPREPRVPRAPRDPRVPRDPRDPRQPRSPREPRSPREPRSPREPRTPRTPREPRTARGSV

SEQ ID NO: 2 - protein, 69 aa, Herpes simplex virus type 1
MPRVPRPPREPREPRVPRAPRDPRVPRDPRDPRQPRSPREPRSPREPRSPREPRTPRTPREPRTARGSV

SEQ ID NO: 3 - protein, 91 aa, Unknown
  Description of Unknown Myc inhibitor sequence
MTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 4 - DNA, 78 bp, Artificial Sequence
  Description of Artificial Sequence Synthetic primer
cttgttctttttgcagaagctcagaataaacgctcaactttggccaccatggaagatgccaaaaacattaagaagggc

SEQ ID NO: 5 - DNA, 157 bp, Artificial Sequence
  Description of Artificial Sequence Synthetic primer
agaatgtgaagaaactttctttttattaggagcagatacgaatggctacattttgggggacaacattttgtaaagtgtaagttggtattatgtagcttagagactccattcgggtgttcttgaggctggtctatcattacacggcgatcttgccgcc

SEQ ID NO: 6 - DNA, 45 bp, Artificial Sequence
  Description of Artificial Sequence Synthetic primer
gaatttaatacgactcactataaggcttgttctttttgcagaagc

SEQ ID NO: 7 - DNA, 149 bp, Artificial Sequence
  Description of Artificial Sequence Synthetic primer
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 8 - RNA, 1980 nt, Artificial Sequence
  Description of Artificial Sequence Synthetic polynucleotide
aggcuuguucuuuuugcagaagccuuguucuuuuugcagaagcucagaauaaacgcucaacuuuggccaccauggaagaugccaaaaacauuaagaagggcccagcgccauucuacccacucgaagacgggaccgccggcgagcagcugcacaaagccaugaagcgcuacgcccuggugcccggcaccaucgccuuuaccgacgcacauaucgagguggacauuaccuacgccgaguacuucgagaugagcguucggcuggcagaagcuaugaagcgcuaugggcugaauacaaaccaucggaucguggugugcagcgagaauagcuugcaguucuucaugcccguguugggugcccuguucaucgguguggcuguggccccagcuaacgacaucuacaacgagcgcgagcugcugaacagcaugggcaucagccagcccaccgucguauucgugagcaagaaagggcugcaaaagauccucaacgugcaaaagaagcuaccgaucauacaaaagaucaucaucauggauagcaagaccgacuaccagggcuuccaaagcauguacaccuucgugacuucccauuugccacccggcuucaacgaguacgacuucgugcccgagagcuucgaccgggacaaaaccaucgcccugaucaugaacaguaguggcaguaccggauugcccaagggcguagcccuaccgcaccgcaccgcuuguguccgauucagucaugcccgcgaccccaucuucggcaaccagaucauccccgacaccgcuauccucagcguggugccauuucaccacggcuucggcauguucaccacgcugggcuacuugaucugcggcuuucgggucgugcucauguaccgcuucgaggaggagcuauucuugcgcagcuugcaagacuauaagauucaaucugcccugcuggugcccacacuauuuagcuucuucgcuaagagcacucucaucgacaaguacgaccuaagcaacuugcacgagaucgccagcggcggggcgccgcucagcaaggagguaggugaggccguggccaaacgcuuccaccuaccaggcauccgccagggcuacggccugacagaaacaaccagcgccauucugaucacccccgaaggggacgacaagccuggcgcaguaggcaagguggugcccuucuucgaggcuaaggugguggacuuggacaccgguaagacacugggugugaaccagcgcggcgagcugugcguccguggccccaugaucaugagcggcuacguuaacaaccccgaggcuacaaacgcucucaucgacaaggacggcuggcugcacagcggcgacaucgccuacugggacgaggacgagcacuucuucaucguggaccggcugaagagccugaucaaauacaagggcuaccagguagccccagccgaacuggagagcauccugcugcaacaccccaacaucuucgacgccggggucgccggccugcccgacgacgaugccggcgagcugcccgccgcagucgucgugcuggaacacgguaaaaccaugaccgagaaggagaucguggacuauguggccagccagguuacaaccgccaagaagcugcgcggugguguuguguucguggacgaggugccuaaaggacugaccggcaaguuggacgcccgcaagauccgcgagauucucauuaaggccaagaagggcggcaagaucgccguguaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 670 bp, Artificial Sequence
  Description of Artificial Sequence Synthetic polynucleotide
cttgttctttttgcagaagctcagaataaacgctcaactttggccaccatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataacgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccgcgttccccaagggagccccggtctccccgtgaaccccggtctcccagggagccccggaccccacgcaccccccgcgaaccacgtacggctcgcggttctgtataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 10 - RNA, 813 nt, Artificial Sequence
  Description of Artificial Sequence Synthetic polynucleotide
aggcuuguucuuuuugcagaagccuuguucuuuuugcagaagcucagaauaaacgcucaacuuuggccaccaugagccagacccaacccccggccccaguugggccgggcgacccagauguuuacuuaaaaggcgugccguccgccggcaugcaccccagagguguucacgcaccucgaggacacccgcgcaugaucuccggacccccgcaacggggugauaacgaucaagcggcggggcaauguggagauucgggucuacuacgagucggugcggacacuacgaucucgaagccaucugaagccguccgaccgccaacaauccccaggacaccgcguguuccccgggagccccggguuccgcgaccaccccgagaaccuagggaacccagaguaccgcgagcucccagagaccccaggguaccgcgugaccccagggauccacgacaaccgcguuccccaagggagccccggucuccccgugaaccccggucucccagggagccccggaccccacgcaccccccgcgaaccacguacggcucgcgguucuguauaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 463 bp, Artificial Sequence
  Description of Artificial Sequence Synthetic polynucleotide
taacttgttctttttgcagaagctcagaataaacgctcaactttggccaccatgaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagagacgcaaaagctcatttctgaaatcgacttgttgcggaaacaaaacgaacagttgaaacacaaacttgaacagctacggaactcttgtgcgtaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 12 - DNA, 463 bp, Artificial Sequence
  Description of Artificial Sequence Synthetic polynucleotide
taacttgttctttttgcagaagctcagaataaacgctcaactttggccaccatgctagtcaacctccttaaggacgagcgtcgggcgaacatccgcgccaaacggcacttgacactgcaggtccagatcaatcggcaacaagccgtcaaggaaacctctgtagcattgaggacggagttgcacaacctgaggctaatcacattgaataaagaaccgcaatttgttaaagccaaaaaccgatcctttatcgacagggagcaggagtctaaattgtgtgaaaacgcaaagtaccagagcgagcttcccaagattaaagaagaggaataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 13 - DNA, 463 bp, Artificial Sequence
  Description of Artificial Sequence Synthetic polynucleotide
taacttgttctttttgcagaagctcagaataaacgctcaactttggccaccatgaacaagaactctatccggcaatctcttaaaaacgttcggttggacgaagtcgcaaacgcacacttgcaacagagggaagtcaaaccgatcgacatcaagcgcctgagcaaagccgagaacaaatttcaatacttgagggtcgaaaagcagacactattggccgagcccaaagagtgtacggagcgtaataaggagtttcaggtagaactgaggacacgagagcagcgggcccacttgattaccctacttctcgaaatcaaagcgaattcctaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 14 - RNA, 603 nt, Artificial Sequence
  Description of Artificial Sequence Synthetic polynucleotide
aggcuuguucuuuuugcagaagccuuguucuuuuugcagaagcucagaauaaacgcucaacuuuggccaccaugaccgaggagaaugucaagaggcgaacacacaacgucuuggagcgccagaggaggaacgagcuaaaacggagcuuuuuugcccugcgugaccagaucccggaguuggaaaacaaugaaaaggcccccaagguaguuauccuuaaaaaagccacagcauacauccuguccguccaagcagagacgcaaaagcucauuucugaaaucgacuuguugcggaaacaaaacgaacaguugaaacacaaacuugaacagcuacggaacucuugugcguaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 603 nt, Artificial Sequence
  Description of Artificial Sequence Synthetic polynucleotide
aggcuuguucuuuuugcagaagccuuguucuuuuugcagaagcucagaauaaacgcucaacuuuggccaccaugcuagucaaccuccuuaaggacgagcgucgggcgaacauccgcgccaaacggcacuugacacugcagguccagaucaaucggcaacaagccgucaaggaaaccucuguagcauugaggacggaguugcacaaccugaggcuaaucacauugaauaaagaaccgcaauuuguuaaagccaaaaaccgauccuuuaucgacagggagcaggagucuaaauugugugaaaacgcaaaguaccagagcgagcuucccaagauuaaagaagaggaauaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - RNA, 603 nt, Artificial Sequence
  Description of Artificial Sequence Synthetic polynucleotide
aggcuuguucuuuuugcagaagccuuguucuuuuugcagaagcucagaauaaacgcucaacuuuggccaccaugaacaagaacucuauccggcaaucucuuaaaaacguucgguuggacgaagucgcaaacgcacacuugcaacagagggaagucaaaccgaucgacaucaagcgccugagcaaagccgagaacaaauuucaauacuugagggucgaaaagcagacacuauuggccgagcccaaagaguguacggagcguaauaaggaguuucagguagaacugaggacacgagagcagcgggcccacuugauuacccuacuucucgaaaucaaagcgaauuccuaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 25 bp, Artificial Sequence
  Description of Artificial Sequence Synthetic oligonucleotide
ttttttttttttttttttttttttt